(19) United States
(12) Patent Application Publication
(10) Pub. No.: US 2004/0097376 A1
(43) Pub. Date: May 20, 2004

(54) Substituted aryl ketones and their use as herbicides

(76) Inventors: Klaus-Helmut Muller, Dusseldorf (DE); Stefan Herrmann, Langenfeld (DE); Dorothee Hoischen, Dusseldorf (DE); Stefan Lehr, Langenfeld (DE); Hans-Georg Schwarz, Langenfeld (DE); Otto Schallner, Monheim (DE); Mark Wilhelm Drewes, Langenfeld (DE); Peter Dahmen, Neuss (DE); Dieter Feucht, Monheim (DE); Rolf Pontzen, Leichlingen (DE)

Correspondence Address: BAYER CROPSCIENCE LP, Patent Department, 100 BAYER ROAD, PITTSBURGH, PA, 15205-9741, US

(21) Appl. No.: 10/343,070

(22) PCT Filed: Jul. 17, 2001
(86) PCT No.: PCT/EP01/08225

(30) Foreign Application Priority Data
Jul. 29, 2000 (DE) 100 37 149.3

(51) Int. Cl. A01N 43/66; C07D 413/02; C 07D 4 3/02

(52) U.S. Cl. 504/230; 544/211; 544/210

(57) ABSTRACT

The invention relates to novel substituted aryl ketones of the formula (I)

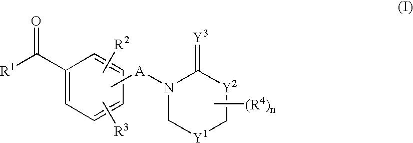

in which

n, A, R¹, R², R³, R⁴, y¹, y² and y³ have the meaning indicated in the description, and processes for preparing the compounds and their use as herbicides.

The invention relates to novel substituted aryl ketones, processes for their preparation and their use as herbicides.

It is already known that certain substituted aryl ketones have herbicidal properties (cf. EP-A-090262, EP-A-135 191, EP-A-186 118, EP-A-186 119, EP-A-186 120, EP-A-319 075, EP-A-352 543, EP-A-418 175, EP-A-487 357, EP-A-527 036, EP-A-527 037, EP-A-560 483, EP-A-609 797, EP-A-609 798, EP-A-625 505, EP-A-625 508, EP-A-636 622, U.S. Pat. No. 5,804,532, U.S. Pat. No. 5,834,402, U.S. Pat. No. 5,846,906, U.S. Pat. No. 5,863,865, WO-A-96/26192, WO-A-96/26193, WO-A-96/26200, WO-A-96/26206, WO-A-97/27187, WO-A-97/35850, WO-A-97/41105, WO-A-97/41116, WO-A-97/41117, WO-A-97/41118, WO-A-97/43270, WO-A-97/46530, WO-A-98/28981, WO-A-98/31681, WO-A-98/31682, WO-A-99/03856, WO-A-99/07688, WO-A-99/10327, WO-A-99/10328, WO-A-00/05221). However, the effect of these compounds is not satisfactory in all respects.

The novel substituted aryl ketones of the formula (I)

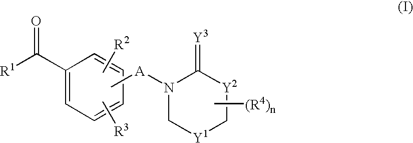

in which

n represents the numbers 0, 1, 2, 3 or 4,

A represents alkanediyl (alkylene),

R¹ represents one of the following groups.

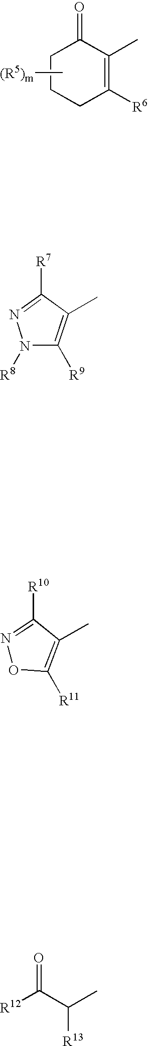

where

m represents the numbers 0 to 6,

R⁵ represents halogen or represents in each case optionally substituted alkyl, alkylthio or aryl or—in the case where m represents 2—optionally also together with a second R⁵ radical represents oxygen or alkanediyl (alkylene),

R⁶ represents hydroxyl, formyloxy, halogen, or represents in each case optionally substituted alkoxy, alkylthio, alkylsulphinyl, alkylsulphonyl, alkylcarbonyloxy, alkoxycarbonyloxy, alkylaminocarbonyloxy, alkylsulphonyloxy, alkenyloxy, alkinyloxy, aryloxy, arylthio, arylsulphinyl, arylsulphonyl, arylcarbonyloxy, arylcarbonylalkoxy, arylsulphonyloxy, arylalkoxy, arylalkylthio, arylalkylsulphinyl or arylalkylsulphonyl,

R⁷ represents hydrogen, cyano, carbamoyl, thiocarbamoyl, halogen, or represents in each case optionally substituted alkyl, alkoxy, alkylthio, alkylsulphinyl, alkylsulphonyl, alkoxycarbonyl or cycloalkyl,

R⁸ represents hydrogen or represents in each case optionally substituted alkyl, alkenyl, alkinyl, cycloalkyl, cycloalkylalkyl, aryl or arylalkyl,

R⁹ represents hydroxyl, formyloxy or represents in each case optionally substituted alkoxy, alkylcarbonyloxy, alkoxycarbonyloxy, alkylaminocarbonyloxy, alkylsulphonyloxy, alkenyloxy, alkinyloxy, arylalkoxy, arylcarbonyloxy, arylcarbonylalkoxy or arylsulphonyloxy,

R¹⁰ represents hydrogen, cyano, carbamoyl, thiocarbamoyl, halogen or represents in each case optionally substituted alkyl, alkylcarbonyl, alkoxy, alkoxycarbonyl, alkylthio, alkylsulphinyl or alkylsulphonyl,

R¹¹ represents hydrogen or represents in each case optionally substituted alkyl or cycloalkyl,

R¹² represents hydrogen or represents in each case optionally substituted alkyl or cycloalkyl, and

R¹³ represents hydrogen, cyano, carbamoyl, halogen, or represents in each case optionally substituted alkyl, alkoxy, alkoxycarbonyl, alkylthio, alkylsulphinyl or alkylsulphonyl,

R² represents hydrogen, nitro, cyano, carboxyl, carbamoyl, thiocarbamoyl, halogen or represents in each case optionally substituted alkyl, alkoxy, alkylthio, alkylsulphinyl, alkylsulphonyl, alkylamino, dialkylamino or dialkylaminosulphonyl,

R³ represents hydrogen, nitro, cyano, carboxyl, carbamoyl, thiocarbamoyl, halogen or represents in each case optionally substituted alkyl, alkoxy, alkylthio, alkylsulphinyl, alkylsulphonyl, alkylamino, dialkylamino or dialkylaminosulphonyl, and

R⁴ represents optionally substituted alkyl,

Y¹ represents a single bond, O (oxygen), S (sulphur), N—Z or optionally substituted (by R⁴) alkanediyl,

Y² represents S (sulphur) or N—Z,

Y³ represents N—Y⁴ or C(Y⁴,Y⁵) or—in the case where Y¹ represents O (oxygen), S (sulphur) or N—Z—also represents O (oxygen) or S (sulphur),

Y⁴ represents cyano, nitro or represents in each case optionally substituted alkylcarbonyl, alkylsulphonyl, arylcarbonyl or arylsulphonyl,

Y⁵ represents hydrogen, cyano, nitro or represents in each case optionally substituted alkylcarbonyl, alkylsulphonyl, arylcarbonyl or arylsulphonyl,

Z represents hydrogen or represents in each case optionally substituted alkyl, alkenyl or alkinyl,

including all possible tautomeric and/or stereoisomeric forms of the compounds of the general formula (I) and the possible salts and acid or base adducts of the compounds of the general formula (I)

have now been found.

In the definitions, the hydrocarbon chains such as alkyl or alkanediyl—also in combination with heteroatoms such as in alkoxy—are in each case straight-chain or branched.

Where the compounds of the general formula can exist in various stereoisomeric forms, the invention includes each of the possible stereoisomeric forms.

Preferred substituents and preferred ranges for the radicals present in the formulae detailed above and hereinafter are defined below.

n preferably represents the numbers 0, 1, 2 or 3.

A preferably represents alkanediyl (alkylene) having 1 to 6 carbon atoms.

R¹ preferably represents one of the following groups

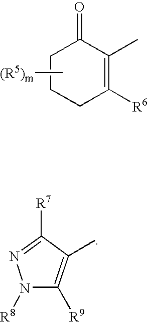

R² preferably represents hydrogen, nitro, cyano, carboxyl, carbamoyl, thiocarbamoyl, halogen or represents in each case optionally cyano-, halogen-, C₁-C₄-alkoxy-, C₁-C₄-alkylthio-, C₁-C₄-alkylsulphinyl- or C₁-C₄-alkyl-sulphonyl-substituted alkyl, alkoxy, alkylthio, alkylsulphinyl, alkylsulphonyl, alkylamino, dialkylamino or dialkylaminosulphonyl having, in each case, 1 to 6 carbon atoms in the alkyl groups.

R³ preferably represents hydrogen, nitro, cyano, carboxyl, carbamoyl, thiocarbamoyl, halogen or represents in each case optionally cyano-, halogen-, C₁-C₄-alkoxy-, C₁-C₄-alkylthio-, C₁-C₄-alkylsulphinyl- or C₁-C₄-alkyl-sulphonyl-substituted alkyl, alkoxy, alkylthio, alkylsulphinyl, alkylsulphonyl, alkylamino, dialkylamino or dialkylaminosulphonyl having, in each case, 1 to 6 carbon atoms in the alkyl groups.

R⁴ preferably represents optionally cyano- or halogen-substituted alkyl having 1 to 6 carbon atoms.

m preferably represents the numbers 0 to 4.

R⁵ preferably represents halogen, represents in each case optionally halogen-substituted alkyl or alkylthio having, in each case, 1 to 6 carbon atoms or represents phenyl or—in the case where m represents 2—optionally also together with a second R⁵ radical represents oxygen or alkanediyl (alkylene) having 3 to 5 carbon atoms.

R⁶ preferably represents hydroxyl, formyloxy, halogen, represents in each case optionally halogen-substituted alkoxy, alkylthio, alkylsulphinyl, alkylsulphonyl, alkylcarbonyloxy, alkoxycarbonyloxy, alkylaminocarbonyloxy or alkylsulphonyloxy having, in each case, 1 to 6 carbon atoms in the alkyl groups, represents in each case optionally halogen-substituted alkenyloxy or alkinyloxy having, in each case, 3 to 6 carbon atoms, represents in each case optionally nitro-, cyano-, halogen-, C₁-C₄-alkyl-, C₁-C₄-halogenoalkyl-, C₁-C₄-alkoxy-, C₁-C₄-halogenoalkoxy-, C₁-C₄-alkylthio-, C₁-C₄-halogenoalkylthio-, C₁-C₄-alkylsulphinyl-, C₁-C₄-halogenoalkylsulphinyl-, C₁-C₄-alkylsulphonyl- or C₁-C₄-halogenoalkylsulphonyl-substituted aryloxy, arylthio, arylsulphinyl, arylsulphonyl, arylcarbonyloxy, arylcarbonylalkoxy, arylsulphonyloxy, arylalkoxy, arylalkylthio, arylalkylsulphinyl or arylalkylsulphonyl having, in each case, 6 or 10 carbon atoms in the aryl group and, where appropriate, 1 to 4 carbon atoms in the alkyl moiety.

R⁷ preferably represents hydrogen, cyano, carbamoyl, thiocarbamoyl, halogen, represents in each case optionally halogen-substituted alkyl, alkoxy, alkylthio, alkylsulphinyl, alkylsulphonyl, alkoxycarbonyl having, in each case, 1 to 6 carbon atoms in the alkyl groups, or represents optionally cyano-, halogen- or C₁-C₄-alkyl-substituted cycloalkyl having 3 to 6 carbon atoms.

R⁸ preferably represents hydrogen, represents optionally cyano-, halogen- or C₁-C₄-alkoxy-substituted alkyl having 1 to 6 carbon atoms, represents in each case optionally cyano- or halogen-substituted alkenyl or alkinyl having, in each case, 3 to 6 carbon atoms, represents in each case optionally cyano-, halogen- or C₁-C₄-alkyl-substituted cycloalkyl or cycloalkylalkyl having, in each case, 3 to 6 carbon atoms in the cycloalkyl group and, where appropriate, 1 to 4 carbon atoms in the alkyl moiety, or represents in each case optionally nitro-, cyano-, halogen-, C₁-C₄-alkyl-, C₁-C₄-halogenoalkyl-, C₁-C₄-alkoxy-, C₁-C₄-halogenoalkoxy-, C₁-C₄-alkylthio-, C₁-C₄-halogenoalkylthio-, C₁-C₄-alkylsulphinyl-, C₁-C₄-halogenoalkylsulphinyl-, C₁-C₄-alkylsulphonyl- or C₁-C₄-halogenoalkylsulphonyl-substituted aryl or arylalkyl having, in each case, 6 or 10 carbon atoms in the aryl group and, where appropriate, 1 to 4 carbon atoms in the alkyl moiety.

R⁹ preferably represents hydroxyl, formyloxy, represents in each case optionally cyano-, halogen- or C₁-C₄-alkoxy-substituted alkoxy, alkylcarbonyloxy, alkoxycarbonyloxy, alkylaminocarbonyloxy or alkylsulphonyloxy having, in each case, 1 to 6 carbon atoms in the alkyl groups, represents in each case optionally cyano- or halogen-substituted alkenyloxy or alkinyloxy having, in each case, 3 to 6 carbon atoms, or represents in each case optionally nitro-, cyano-, halogen-, C₁-C₄-alkyl-, C₁-C₄-halogenoalkyl-, C₁-C₄-alkoxy-, C₁-C₄-halogenoalkoxy-, C₁-C₄-alkylthio-, C₁-C₄-halogenoalkylthio-, C₁-C₄-alkylsulphinyl-, C₁-C₄-halogenoalkylsulphinyl-, C₁-C₄-alkylsulphonyl- or C₁-C₄-halogenoalkylsulphonyl-substituted arylalkoxy, arylcarbonyloxy, arylcarbonylalkoxy or arylsulphonyloxy having 6 or 10 carbon atoms in the aryl group and, where appropriate, 1 to 4 carbon atoms in the alkyl moiety.

R¹⁰ preferably represents hydrogen, cyano, carbamoyl, thiocarbamoyl, halogen, or represents in each case optionally cyano-, halogen- or C₁-C₄-alkoxy-substituted alkyl, alkylcarbonyl, alkoxy, alkoxycarbonyl, alkylthio, alkylsulphinyl or alkylsulphonyl having, in each case, 1 to 6 carbon atoms in the alkyl groups.

R¹¹ preferably represents hydrogen, represents optionally cyano-, halogen- or C₁-C₄-alkoxy-substituted alkyl having 1 to 6 carbon atoms or represents optionally cyano-, halogen- or C₁-C₄-alkyl-substituted cycloalkyl having 3 to 6 carbon atoms.

R¹² preferably represents hydrogen, represents optionally cyano-, halogen- or C₁-C₄-alkoxy-substituted alkyl having 1 to 6 carbon atoms or represents optionally cyano-, halogen- or C₁-C₄-alkyl-substituted cycloalkyl having 3 to 6 carbon atoms.

R¹³ preferably represents hydrogen, cyano, carbamoyl, halogen, or represents optionally in each case cyano-, halogen- or C₁-C₄-alkoxy-substituted alkyl, alkoxy, alkoxycarbonyl, alkylthio, alkylsulphinyl or alkylsulphonyl having, in each case, 1 to 6 carbon atoms in the alkyl groups.

Y¹ preferably represents a single bond, O (oxygen), S (sulphur), N—Z or optionally substituted (by R⁴) alkanediyl having 1 to 4 carbon atoms.

Y³ preferably represents N—Y⁴ or—in the case where Y¹ represents O (oxygen), S (sulphur) or N—Z—also represents O (oxygen) or S (sulphur).

Y⁴ preferably represents cyano, nitro, represents in each case optionally halogen-substituted alkylcarbonyl or alkylsulphonyl having, in each case, up to 6 carbon atoms, or represents in each case optionally nitro-, cyano-, halogen-, C₁-C₄-alkyl-, C₁-C₄-halogenoalkyl-, C₁-C₄-alkoxy- or C₁-C₄-halogenoalkoxy-substituted arylcarbonyl or arylsulphonyl having, in each case, 6 or 10 carbon atoms.

Y⁵ preferably represents hydrogen, cyano, nitro, represents in each case optionally halogen-substituted alkylcarbonyl or alkylsulphonyl having, in each case, up to 6 carbon atoms, or represents in each case optionally nitro-, cyano-, halogen-, C₁-C₄-alkyl-, C₁-C₄-halogenoalkyl-, C₁-C₄-alkoxy- or C₁-C₄-halogenoalkoxy-substituted arylcarbonyl or arylsulphonyl having, in each case, 6 or 10 carbon atoms.

Z preferably represents hydrogen, represents optionally cyano-, halogen- or C₁-C₄-alkoxy-substituted alkyl having 1 to 6 carbon atoms, or represents in each case optionally cyano- or halogen-substituted alkenyl or alkinyl having, in each case, 3 to 6 carbon atoms.

n particularly preferably represents the numbers 0, 1 or 2.

A particularly preferably represents methylene, ethane-1,1-diyl (ethylidene), ethane-1,2-diyl (dimethylene), propane-1,1-diyl (propylidene), propane-1,2-diyl, propane-1,3-diyl (trimethylene), butane-1,2-diyl, butane-1,3-diyl or butane-1,4-diyl.

R² particularly preferably represents hydrogen, nitro, cyano, carboxyl, carbamoyl, thiocarbamoyl, fluorine, chlorine, bromine, iodine, or represents in each case optionally cyano-, fluorine-, chlorine-, methoxy-, ethoxy-, methylthio-, ethylthio-, methylsulphinyl-, ethylsulphinyl-, methylsulphonyl- or ethylsulphonyl-substituted methyl, ethyl, n- or i-propyl, n-, i-, s- or t-butyl, methoxy, ethoxy, n- or i-propoxy, n-, i-, s- or t-butoxy, methylthio, ethylthio, n- or i-propylthio, n-, i-, s- or t-butylthio, methylsulphinyl, ethylsulphinyl, n- or i-propylsulphinyl, methylsulphonyl, ethylsulphonyl, n- or i-propylsulphonyl, methylamino, ethylamino, n- or i-propylamino, n-, i-, s- or t-butylamino, dimethylamino, diethylamino, dimethylaminosulphonyl or diethylaminosulphonyl.

R³ particularly preferably represents hydrogen, nitro, cyano, carboxyl, carbamoyl, thiocarbamoyl, fluorine, chlorine, bromine, iodine, or represents in each case optionally cyano-, fluorine-, chlorine-, methoxy-, ethoxy-, methylthio-, ethylthio-, methylsulphinyl-, ethylsulphinyl- methylsulphonyl- or ethylsulphonyl-substituted methyl, ethyl, n- or i-propyl, n-, i-, s- or t-butyl, methoxy, ethoxy, n- or i-propoxy, n-, i-, s- or t-butoxy, methylthio, ethylthio, n- or i-propylthio, n-, i-, s- or t-butylthio, methylsulphinyl, ethylsulphinyl, n- or i-propylsulphinyl, methylsulphonyl, ethylsulphonyl, n- or i-propylsulphonyl, methylamino, ethylamino, n- or i-propylamino, n-, i-, s- or t-butylamino, dimethylamino, diethylamino, dimethylaminosulphonyl or diethylaminosulphonyl.

R⁴ particularly preferably represents methyl, ethyl, n- or i-propyl, chloromethyl, dichloromethyl, fluoromethyl, difluoromethyl, trifluoromethyl, chlorodifluoromethyl or fluorodichloromethyl.

m particularly preferably represents the numbers 0, 1, 2 or 3.

R⁵ particularly preferably represents fluorine, chlorine, bromine, represents in each case optionally fluorine- and/or chlorine-substituted methyl, ethyl, n- or i-propyl, n-, i-, s- or t-butyl, methylthio, ethylthio, n- or i-propylthio, n-, i-, s- or t-butylthio, or represents phenyl or—in the case where m represents 2—where appropriate also together with a second R⁵ radical represents oxygen, propane-1,3-diyl or butane-1,4-diyl.

R⁶ particularly preferably represents hydroxyl, formyloxy, fluorine, chlorine, represents in each case optionally fluorine- and/or chlorine-substituted methoxy, ethoxy, n- or i-propoxy, n-, i-, s- or t-butoxy, methylthio, ethylthio, n- or i-propylthio, n-, i-, s- or t-butylthio, methylsulphinyl, ethylsulphinyl, n- or i-propylsulphinyl, methylsulphonyl, ethylsulphonyl, n- or i-propylsulphonyl, acetyloxy, propionyloxy, n- or i-butyryloxy, methoxycarbonyloxy, ethoxycarbonyloxy, n- or i-propoxycarbonyloxy, methylaminocarbonyloxy, ethylaminocarbonyloxy, n- or i-propylaminocarbonyloxy, methylsulphonyloxy, ethylsulphonyloxy, n- or i-propylsulphonyloxy, represents in each case optionally fluorine-, chlorine- or bromine-substituted optionally nitro-, cyano-, fluorine-, chlorine-, bromine-, methyl-, ethyl-, n- or i-propyl-, n-, i-, s- or t-butyl-, trifluoromethyl-, methoxy-, ethoxy-, n- or i-propoxy-, n-, i-, s- or t-butoxy-, difluoromethoxy-, trifluoromethoxy-, methylthio-, ethylthio-, n- or i-propylthio-, n-, i-, s- or t-butylthio-, difluoromethylthio-, trifluoromethylthio-, methylsulphinyl-, ethylsulphinyl-, n- or i-propylsulphinyl-, trifluoromethylsulphinyl-, methylsulphonyl-, ethylsulphonyl-, n- or i-propylsulphonyl- or trifluoromethylsulphonyl-substituted phenoxy, phenylthio, phenylsulphinyl, phenylsulphonyl, phenylcarbonyloxy, phenylcarbonylmethoxy, phenylsulphonyloxy, phenylmethoxy, phenylmethylthio, phenylmethylsulphinyl or phenylmethylsulphonyl.

R⁷ particularly preferably represents hydrogen, cyano, carbamoyl, thiocarbamoyl, fluorine, chlorine, bromine, represents in each case optionally fluorine- and/or chlorine-substituted methyl, ethyl, n- or i-propyl, n-, i-, s- or t-butyl, methoxy, ethoxy, n- or i-propoxy, n-, i-, s- or t-butoxy, methylthio, ethylthio, n- or i-propylthio, n-, i-, s- or t-butylthio, methylsulphinyl, ethylsulphinyl, n- or i-propylsulphinyl, methylsulphonyl, ethylsulphonyl, n- or i-propylsulphonyl, methoxycarbonyl, ethoxycarbonyl, n- or i-propoxycarbonyl, or represents in each case optionally cyano-, fluorine-, chlorine-, bromine-, methyl-, ethyl-, n- or i-propyl-substituted cyclopropyl, cyclobutyl, cyclopentyl or cyclohexyl.

R⁸ particularly preferably represents hydrogen, represents in each case optionally cyano-, fluorine-, chlorine-, methoxy- or ethoxy-substituted methyl, ethyl, n- or i-propyl, n-, i-, s- or t-butyl, represents in each case optionally cyano-, fluorine-, chlorine- or bromine-substituted propenyl, butenyl, propinyl or butinyl, represents in each case optionally cyano-, fluorine-, chlorine-, methyl-, ethyl-, n- or i-propyl-substituted cyclopropyl, cyclobutyl, cyclopentyl, cyclohexyl, cyclopropylmethyl, cyclobutylmethyl, cyclopentylmethyl or cyclohexylmethyl or represents in each case optionally nitro-, cyano-, fluorine-, chlorine-, bromine-, methyl-, ethyl-, n- or i-propyl-, n-, i-, s- or t-butyl-, trifluoromethyl-, methoxy-, ethoxy-, n- or i-propoxy-, n-, i-, s- or t-butoxy-, difluoromethoxy-, trifluoromethoxy-, methylthio-, ethylthio-, n- or i-propylthio-, n-, i-, s- or t-butylthio-, difluoromethylthio-, trifluoromethylthio-, methylsulphinyl-, ethylsulphinyl-, n- or i-propylsulphinyl-, trifluoromethylsulphinyl-, methylsulphonyl-, ethylsulphonyl-, n- or i-propylsulphonyl- or trifluoromethylsulphonyl-substituted phenyl, benzyl or phenylethyl.

R⁹ particularly preferably represents hydroxyl, formyloxy, represents in each case optionally cyano-, fluorine-, chlorine-, methoxy-, ethoxy-, n- or i-propoxy-substituted methoxy, ethoxy, n- or i-propoxy, n-, i-, s- or t-butoxy, acetyloxy, propionyloxy, n- or i-butyryloxy, methoxycarbonyloxy, ethoxycarbonyloxy, n- or i-propoxycarbonyloxy, methylaminocarbonyloxy, ethylaminocarbonyloxy, n- or i-propylaminocarbonyloxy, methylsulphonyloxy, ethylsulphonyloxy, n- or i-propylsulphonyloxy, represents in each case optionally cyano-, fluorine-, chlorine- or bromine-substituted propenyloxy, butenyloxy, propinyloxy or butinyloxy, or represents in each case optionally nitro-, cyano-, fluorine-, chlorine-, bromine-, methyl-, ethyl-, n- or i-propyl-, n-, i-, s- or t-butyl-, trifluoromethyl-, methoxy-, ethoxy-, n- or i-propoxy-, n-, i-, s- or t-butoxy-, difluoromethoxy-, trifluoromethoxy-, methylthio-, ethylthio-, n- or i-propylthio-, n-, i-, s- or t-butylthio-, difluoromethylthio-, trifluoromethylthio-, methylsulphinyl-, ethylsulphinyl-, n- or i-propylsulphinyl-, trifluoromethylsulphinyl-, methylsulphonyl-, ethylsulphonyl-, n- or i-propylsulphonyl- or trifluoromethylsulphonyl-substituted phenylmethoxy, phenylcarbonyloxy, phenylcarbonylmethoxy or phenylsulphonyloxy.

R¹⁰ particularly preferably represents hydrogen, cyano, carbamoyl, thiocarbamoyl, fluorine, chlorine, bromine, or represents in each case optionally cyano-, fluorine-, chlorine-, methoxy- or ethoxy-substituted methyl, ethyl, n- or i-propyl, n-, i-, s- or t-butyl, acetyl, propionyl, n- or i-butyryl, methoxy, ethoxy, n- or i-propoxy, n-, i-, s- or t-butoxy, methoxycarbonyl, ethoxycarbonyl, n- or i-propoxycarbonyl, methylthio, ethylthio, n- or i-propylthio, n- i-, s- or t-butylthio, methylsulphinyl, ethylsulphinyl, n- or i-propylsulphinyl, methylsulphonyl, ethylsulphonyl, n- or i-propylsulphonyl.

R¹¹ particularly preferably represents hydrogen, represents in each case optionally cyano-, fluorine-, chlorine-, methoxy- or ethoxy-substituted methyl, ethyl, n- or i-propyl, n-, i-, s- or t-butyl, or represents in each case optionally cyano-, fluorine-, chlorine-, methyl- or ethyl-substituted cyclopropyl, cyclobutyl, cyclopentyl or cyclohexyl.

R¹² particularly preferably represents hydrogen, represents in each case optionally cyano-, fluorine-, chlorine-, methoxy- or ethoxy-substituted methyl, ethyl, n- or i-propyl, n-, i-, s- or t-butyl, or represents in each case optionally cyano-, fluorine-, chlorine-, methyl- or ethyl-substituted cyclopropyl, cyclobutyl, cyclopentyl or cyclohexyl.

R¹³ particularly preferably represents hydrogen, cyano, carbamoyl, fluorine, chlorine, bromine, or represents in each case optionally cyano-, fluorine-, chlorine-, methoxy- or ethoxy-substituted methyl, ethyl, n- or i-propyl, n-, i-, s- or t-butyl, methoxy, ethoxy, n- or i-propoxy, n-, i-, s- or t-butoxy, methoxycarbonyl, ethoxycarbonyl, n- or i-propoxycarbonyl, methylthio, ethylthio, n- or i-propylthio, n-, i-, s- or t-butylthio, methylsulphinyl, ethylsulphinyl, n- or i-propylsulphinyl, methylsulphonyl, ethylsulphonyl, n- or i-propylsulphonyl.

Y¹ particularly preferably represents a single bond, O (oxygen), S (sulphur), N—Z, methylene or dimethylene (ethane-1,2-diyl).

Y³ particularly preferably represents N—CN or—in the case where Y¹ represents O (oxygen) or N—Z—also represents O (oxygen).

Y⁴ particularly preferably represents cyano, nitro, represents in each case optionally fluorine- and/or chlorine-substituted acetyl, propionyl, n- or i-butyryl, methylsulphonyl, ethylsulphonyl, n- or i-propylsulphonyl, or represents in each case optionally nitro-, cyano-, fluorine-, chlorine-, bromine-, methyl-, ethyl-, n- or i-propyl-, n-, i-, s- or t-butyl-, trifluoromethyl-, methoxy-, ethoxy-, n- or i-propoxy-, difluoromethoxy- or trifluoromethoxy-substituted benzoyl or phenylsulphonyl.

Y⁵ particularly preferably represents hydrogen, cyano, nitro, represents in each case optionally fluorine- and/or chlorine-substituted acetyl, propionyl, n- or i-butyryl, methylsulphonyl, ethylsulphonyl, n- or i-propylsulphonyl, or represents in each case optionally nitro-, cyano-, fluorine-, chlorine-, bromine-, methyl-, ethyl-, n- or i-propyl-, n-, i-, s- or t-butyl-, trifluoromethyl-, methoxy-, ethoxy-, n- or i-propoxy-, difluoromethoxy- or trifluoromethoxy-substituted benzoyl or phenylsulphonyl.

Z particularly preferably represents hydrogen, represents in each case optionally cyano-, fluorine-, chlorine-, methoxy- or ethoxy-substituted methyl, ethyl, n- or i-propyl, n-, i-, s- or t-butyl, or represents in each case optionally cyano-, fluorine-, chlorine- or bromine-substituted propenyl, butenyl, propinyl or butinyl.

n very particularly preferably represents the number 0.

A very particularly preferably represents methylene or ethane-1,2-diyl (dimethylene).

R² very particularly preferably represents hydrogen, nitro, cyano, fluorine, chlorine, bromine, iodine, methyl, ethyl, n- or i-propyl, difluoromethyl, trifluoromethyl, dichloromethyl, trichloromethyl, methoxymethyl, methylthiomethyl, methylsulphinylmethyl, methylsulphonylmethyl, methoxy, ethoxy, difluoromethoxy, trifluoromethoxy, methylthio, ethylthio, methylsulphinyl, ethylsulphinyl, methylsulphonyl, ethylsulphonyl or dimethylaminosulphonyl.

R³ very particularly preferably represents hydrogen, nitro, cyano, fluorine, chlorine, bromine, iodine, methyl, ethyl, difluoromethyl, trifluoromethyl, dichloromethyl, trichloromethyl, methoxymethyl, methylthiomethyl, methylsulphinylmethyl, methylsulphonylmethyl, methoxy, ethoxy, difluoromethoxy, trifluoromethoxy, methylthio, ethylthio, methylsulphinyl, ethylsulphinyl, methylsulphonyl, ethylsulphonyl or dimethylaminosulphonyl.

m very particularly preferably represents the numbers 0, 1 or 2.

R⁵ very particularly preferably represents in each case optionally fluorine- and/or chlorine-substituted methyl, ethyl, n- or i-propyl, methylthio, ethylthio, n- or i-propylthio, or represents phenyl or—in the case where m represents 2—where appropriate also together with a second R⁵ radical represents propane-1,3-diyl or butane-1,4-diyl.

R⁶ very particularly preferably represents hydroxyl, represents formyloxy, represents in each case optionally fluorine- and/or chlorine-substituted methoxy, ethoxy, n- or i-propoxy, methylthio, ethylthio, n- or i-propylthio, methylsulphinyl, ethylsulphinyl, methylsulphonyl, ethylsulphonyl, acetyloxy, propionyloxy, n- or i-butyryloxy, methoxycarbonyloxy, ethoxycarbonyloxy, n- or i-propoxycarbonyloxy, methylaminocarbonyloxy, ethylaminocarbonyloxy, n- or i-propylaminocarbonyloxy, methylsulphonyloxy, ethylsulphonyloxy, n- or i-propylsulphonyloxy, represents in each case optionally fluorine- or chlorine-substituted propenyloxy, butenyloxy, propinyloxy or butinyloxy, represents in each case optionally nitro-, cyano-, fluorine-, chlorine-, bromine-, methyl-, ethyl-, n- or i-propyl-, trifluoromethyl-, methoxy-, ethoxy-, n- or i-propoxy-, difluoromethoxy- or trifluoromethoxy-substituted phenoxy, phenylthio, phenylsulphinyl, phenylsulphonyl, phenylcarbonyloxy, phenylcarbonylmethoxy, phenylsulphonyloxy, phenylmethoxy, phenylmethylthio, phenylmethylsulphinyl or phenylmethylsulphonyl.

R⁷ very particularly preferably represents hydrogen, cyano, fluorine, chlorine, bromine, represents in each case optionally fluorine- and/or chlorine-substituted methyl, ethyl, n- or i-propyl, methoxy, ethoxy, n- or i-propoxy, methylthio, ethylthio, n- or i-propylthio, methylsulphinyl, ethylsulphinyl, n- or i-propylsulphinyl, methylsulphonyl, ethylsulphonyl, n- or i-propylsulphonyl, methoxycarbonyl, ethoxycarbonyl, n- or i-propoxycarbonyl, or represents optionally cyano-, fluorine-, chlorine- or methyl-substituted cyclopropyl.

R⁸ very particularly preferably represents hydrogen, represents in each case optionally cyano-, fluorine-, chlorine-, methoxy- or ethoxy-substituted methyl, ethyl, n- or i-propyl, n-, i-, s- or t-butyl, represents in each case optionally fluorine- or chlorine-substituted propenyl, butenyl, propinyl or butinyl, represents in each case optionally fluorine-, chlorine-, methyl- or ethyl-substituted cyclopropyl, or represents in each case optionally fluorine-, chlorine-, bromine-, methyl-, ethyl-, n- or i-propyl-, trifluoromethyl-, methoxy-, ethoxy-, n- or i-propoxy-, difluoromethoxy- or trifluoromethoxy-substituted phenyl or benzyl.

R⁹ very particularly preferably represents hydroxyl, represents formyloxy, represents in each case optionally cyano-, fluorine-, chlorine-, methoxy-, ethoxy-, n- or i-propoxy-substituted methoxy, ethoxy, n- or i-propoxy, acetyloxy, propionyloxy, n- or i-butyryloxy, methoxycarbonyloxy, ethoxycarbonyloxy, n- or i-propoxycarbonyloxy, methylaminocarbonyloxy, ethylaminocarbonyloxy, n- or i-propylaminocarbonyloxy, methylsulphonyloxy, ethylsulphonyloxy, n- or i-propylsulphonyloxy, represents in each case optionally fluorine- or chlorine-substituted propenyloxy, butenyloxy, propinyloxy or butinyloxy, or represents in each case optionally nitro-, cyano-, fluorine-, chlorine-, bromine-, methyl-, ethyl-, n- or i-propyl-, trifluoromethyl-, methoxy-, ethoxy-, n- or i-propoxy-, difluoromethoxy- or trifluoromethoxy-substituted phenylmethoxy, phenylcarbonyloxy, phenylcarbonylmethoxy or phenylsulphonyloxy.

R¹⁰ very particularly preferably represents hydrogen, cyano, fluorine, chlorine, bromine, or represents in each case optionally fluorine-, chlorine-, methoxy- or ethoxy-substituted methyl, ethyl, n- or i-propyl, acetyl, propionyl, n- or i-butyryl, methoxy, ethoxy, n- or i-propoxy, methoxycarbonyl, ethoxycarbonyl, n- or i-propoxycarbonyl, methylthio, ethylthio, n- or i-propylthio, methylsulphinyl, ethylsulphinyl, methylsulphonyl or ethylsulphonyl.

R¹¹ very particularly preferably represents hydrogen, represents in each case optionally fluorine-, chlorine-, methoxy- or ethoxy-substituted methyl, ethyl, n- or i-propyl, or represents optionally fluorine-, chlorine-, methyl- or ethyl-substituted cyclopropyl.

R¹² very particularly preferably represents hydrogen, represents in each case optionally fluorine-, chlorine-, methoxy- or ethoxy-substituted methyl, ethyl, n- or i-propyl, n-, i-, s- or t-butyl, or represents optionally cyano-, fluorine-, chlorine-, methyl- or ethyl-substituted cyclopropyl.

R¹³ very particularly preferably represents hydrogen, cyano, fluorine, chlorine, bromine, or represents in each case optionally fluorine-, chlorine-, methoxy- or ethoxy-substituted methyl, ethyl, n- or i-propyl, methoxy, ethoxy, n- or i-propoxy, methoxycarbonyl, ethoxycarbonyl, n- or i-propoxycarbonyl, methylthio, ethylthio, n- or i-propylthio, methylsulphinyl, ethylsulphinyl, n- or i-propylsulphinyl, methylsulphonyl, ethylsulphonyl, n- or i-propylsulphonyl.

Y¹ very particularly preferably represents a single bond, O (oxygen), CH₂ (methylene) or N—Z.

Y³ very particularly preferably represents N—CN.

Z very particularly preferably represents hydrogen, methyl, ethyl, n- or i-propyl.

Compounds of the formula (I) which are preferred according to the invention are those in which a combination of the meanings listed above as preferred is present.

Compounds of the formula (I) which are particularly preferred according to the invention are those in which a combination of the meanings listed above as particularly preferred is present.

Compounds of the formula (I) which are very particularly preferred according to the invention are those in which a combination of the meanings listed above as very particularly preferred is present.

The compounds of the formulae (I-1) to (I-3) are especially emphasized:

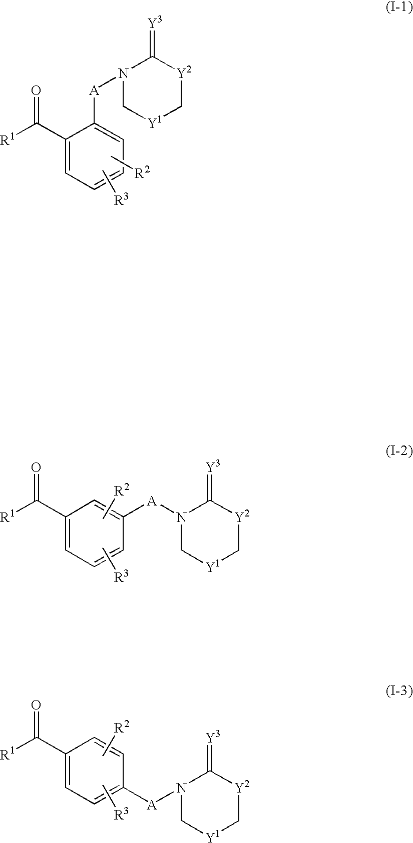

A, R¹, R², R³, Y¹, Y² and Y³ therein each have the meanings indicated above as very particularly preferred.

Further compounds to be especially emphasized are those of the general formulae (I-2)to (I-2D):

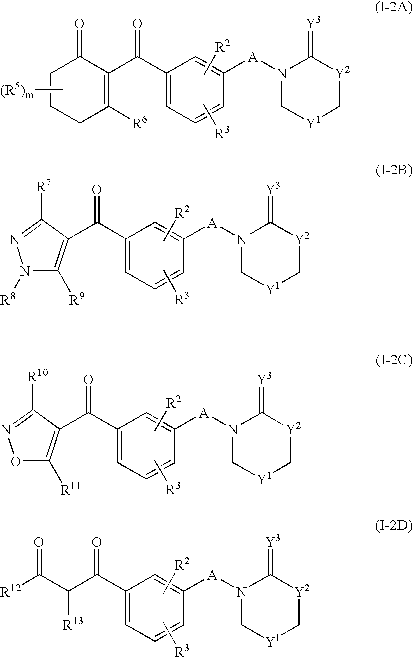

m, A, R², R³, R⁵, R⁶, R⁷, R⁸, R⁹, R¹⁰, R¹¹, R¹², R¹³, Y¹, Y² and Y³ therein each have the meanings indicated above as very particularly preferred.

Those of the compounds of the formulae (I-2A) to (I-2D) in which Y¹ represents a single bond or methylene, and Y³ represents N—CN, are to be very especially emphasized.

The radical definitions generally listed above or listed in preferred ranges apply both to the final products of the formula (I) and correspondingly to the starting materials or intermediates required for the preparation in each case. These radical definitions can be combined together, that is to say also between the preferred range indicated, as desired.

The novel substituted aryl ketones of the formula (I) are distinguished by strong and selective herbicidal activity.

The novel substituted aryl ketones of the formula (I) are obtained when

(a) substituted benzoic esters of the formula (II)

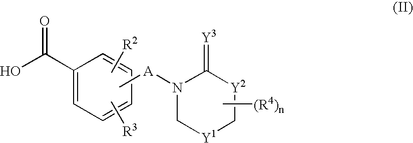

in which

n, A, R², R³, R⁴, Y¹, Y² and Y³ have the meaning indicated above,

or reactive derivatives thereof, such as, for example, corresponding acid halides, acid cyanides or esters

are reacted with compounds of the general formula (III)

R¹—H (III)

in which

R¹ has the meaning indicated above,

where appropriate in the presence of a dehydrating agent, where appropriate in the presence of one or more reaction auxiliaries, and where appropriate in the presence of one or more diluents,

or when

(b) substituted benzoyl ketones of the general formula (Ia)

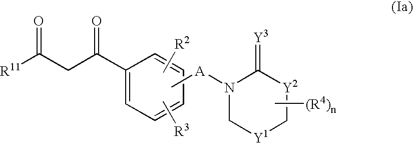

in which

n, A, R², R³, R⁴, R¹¹, Y¹, Y² and Y³ have the meaning indicated above,

are reacted with an orthoformic ester or with an N,N-dimethylformamide acetal or with a cyanoformic ester or with carbon disulphide and an alkylating agent and subsequently with hydroxylamine or an acid adduct thereof

where appropriate in the presence of one or more reaction auxiliaries and where appropriate in the presence of one or more diluents,

and, where appropriate, after processes (a) or (b) according to the invention have been carried out, the compounds of the general formula (I) which have been obtained in this way within the framework of the definition of substituents are subjected to substitution, oxidation or reduction reactions in a conventional way, and/or the compounds of the general formula (I) are converted into salt-like compounds in a conventional way.

If, for example, 4-chloro-2-fluoro-3-[(3,5-dimethyl-2-nitroiminotetrahydro-1,3,5-triazin-1(2H)-yl)methyl]benzoic acid and 1,3-dimethyl-5-hydroxypyrazole are used as starting materials, the course of the reaction in process (a) according to the invention can be outlined by the following formula diagram:

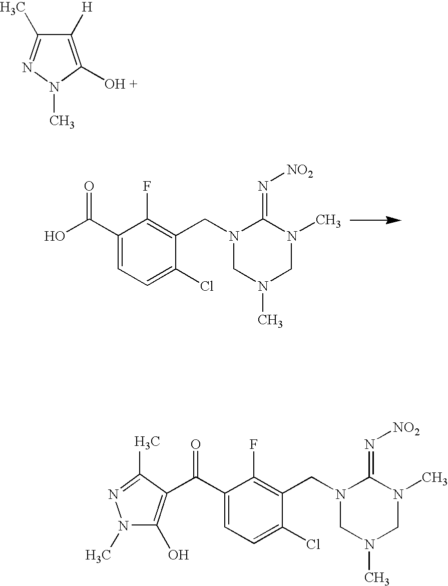

If, for example 3-[2-chloro-3-(3-cyclopropyl-3-oxopropanoyl)-6-methylbenzyl]-1,3-thiazolidin-2-ylidenecyanamide, methyl cyanoformate and hydroxylamine are used as starting materials, the course of the reaction in process (b) according to the invention can be outlined by the following formula diagram:

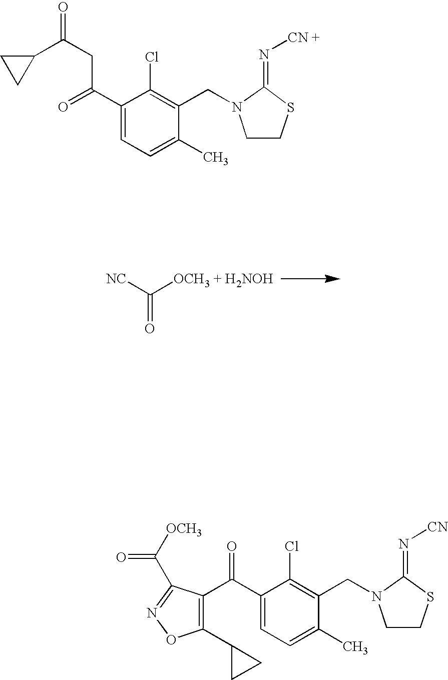

The substituted benzoic acids to be used as starting materials in process (a) according to the invention for preparing compounds of the general formula (a) are generally defined by formula (II). The meanings of n, A, R², R³, R⁴, Y¹, Y² and Y³ in the general formula (II) are preferably those which have already been indicated above in connection with the description of the compounds of the general formula (I) according to the invention as being preferred, particularly preferred or very particularly preferred for n, A, R², R³, R⁴, Y¹, Y² and Y³.

The starting materials of the general formula (II) are—like their reactive derivatives referred to above—not yet known from the literature; as novel substances, the present application also relates to them.

The novel substituted benzoic acids of the general formula (II)—or else their derivatives, such as their esters, especially the methyl esters and ethyl esters—are obtained when

(α) halogenoalkylbenzoic esters of the general formula (IV)

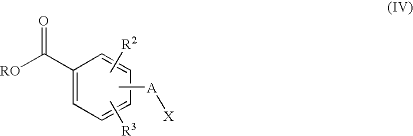

in which

A, R² and R³ have the meaning indicated above,

R represents alkyl, in particular methyl or ethyl, and

X represents halogen, in particular fluorine, chlorine or bromine, or alkylsulphonyloxy, in particular methylsulphonyloxy or ethylsulphonyloxy,

are reacted with compounds of the general formula (V)

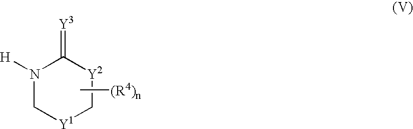

in which

n, R⁴, Y¹, Y² and Y³ have the meaning indicated above,

where appropriate in the presence of an acid acceptor such as, for example, potassium carbonate or sodium hydride, where appropriate in the presence of a diluent such as, for example, acetone or tetrahydrofuran, at temperatures between 0° C. and 100° C. (compare the Preparation Examples),

or when

7- to prepare compounds of the formula (II) in which Y¹ represents O (oxygen) or N—Z and Y² represents N—Z—

(β) halogenoalkylbenzoic esters of the general formula (IV)—above—are reacted in a first step with compounds of the general formula (VI)

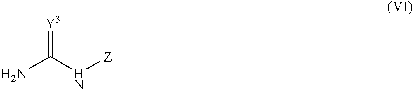

in which

Y³ and Z have the meaning indicated above,

where appropriate in the presence of an acid acceptor such as, for example, potassium carbonate or sodium hydride, where appropriate in the presence of a diluent such as, for example, acetone or tetrahydrofuran, at temperatures between 0° C. and 100° C., and the ureidoalkylbenzoic esters of the general formula (VII) which are formed thereby

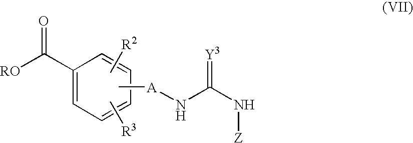

in which

A, R, R², R³, Y³ and Z have the meaning indicated above,

are reacted with formaldehyde or paraformaldehyde, where appropriate in the presence of a reaction auxiliary such as, for example, triethylamine, and where appropriate in the presence of one or more diluents such as, for example, toluene or 1,4-dioxane, and where appropriate with an amine of the general formula H₂N—Z at temperatures between 0° C. and 150° C. (compare Bull. Chem. Soc. Japan 62 (1989), 3721-3733, EP-A-428 941, EP-A-580 553),

and, where appropriate, the substituted benzoic esters of the general formula (IIa) obtained by process variants (α) or (β)

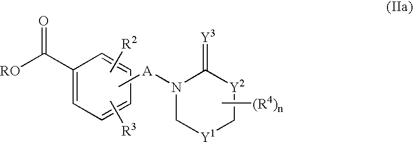

in which

n, A, R, R², R³, R⁴, Y¹, Y² and Y³ have the meaning indicated above,

are hydrolysed in a conventional way, that is to say reacted with water in the presence of a base such as, for example, potassium hydroxide or sodium hydroxide, and where appropriate in the presence of one or more diluents such as, for example, 1,4-dioxane, cyclohexane and 2-methoxyethanol, at temperatures between 0° C. and 100° C. (compare the Preparation Examples).

The other compounds to be used as starting materials in process (a) according to the invention for preparing compounds of the general formula (I) are generally defined by formula (III). The meaning of R¹ in the general formula (III) is preferably that which has already been indicated above in connection with the description of the compounds of the general formula (I) according to the invention as being preferred, particularly preferred or very particularly preferred for R¹.

The starting materials of the general formula (III) are known organic compounds.

The substituted benzoyl ketones to be used as starting materials in process (b) according to the invention for preparing compounds of the general formula (I) are generally defined by formula (Ia). The meanings of n, A, R², R³, R⁴, R¹¹, Y¹, Y² and Y³ in the general formula (Ia) are preferably those which have already been indicated above in connection with the description of the compounds of the general formula (I) according to the invention as being preferred, particularly preferred or very particularly preferred for n, A, R², R³, R⁴, R¹¹, Y¹, Y² and Y³.

The starting materials of the general formula (Ia) are novel compounds according to the invention; they can be prepared by process (a) according to the invention.

Process (a) according to the invention for preparing the novel substituted aryl ketones of the general formula (I) is carried out where appropriate with the use of a dehydrating agent. Suitable for this purpose are the chemicals normally suitable for binding water.

Dicyclohexylcarbodiimide and carbonylbisimidazole are mentioned as examples thereof.

Dicyclohexylcarbodiimide is mentioned as particularly suitable dehydrating agent.

Processes (a) and (b) according to the invention are preferably carried out with the use of one or more reaction auxiliaries. Reaction auxiliaries suitable for processes (a) and (b) according to the invention are generally the conventional inorganic or organic bases or acid acceptors. These preferably include alkali metal or alkaline earth metal acetates, amides, carbonates, bicarbonates, hydrides, hydroxides or alkanolates such as, for example, sodium, potassium or calcium acetate, lithium, sodium, potassium or calcium amide, sodium, potassium or calcium carbonate, sodium, potassium or calcium bicarbonate, lithium, sodium, potassium or calcium hydride, lithium, sodium, potassium or calcium hydroxide, sodium or potassium methanolate, ethanolate, n- or i-propanolate, n-, i-, s- or t-butanolate; furthermore basic organic nitrogen compounds such as, for example, trimethylamine, triethylamine, tripropylamine, tributylamine, ethyldiisopropylamine, N,N-dimethylcyclohexylamine, dicyclohexylamine, ethyldicyclohexylamine, N,N-dimethylaniline, N,N-dimethylbenzylamine, pyridine, 2-methyl-, 3-methyl-, 4-methyl-, 2,4-dimethyl-, 2,6-dimethyl-, 3,4-dimethyl- and 3,5-dimethylpyridine, 5-ethyl-2-methylpyridine, 4-dimethylaminopyridine, N-methylpiperidine, 1,4-diazabicyclo[2.2.2]octane (DABCO), 1,5-diazabicyclo[4.3.0]-non-5-ene (DBN) or 1,8-diazabicyclo[5.4.0]-undec-7-ene (DBU).

Further reaction auxiliaries suitable for processes (a) and (b) according to the invention are also phase-transfer catalysts. Examples of such catalysts which may be mentioned are:

tetrabutylammonium bromide, tetrabutylammonium chloride, tetraoctylammonium chloride, tetrabutylammonium bisulphate, methyltrioctylammonium chloride, hexadecyltrimethylammonium chloride, hexadecyltrimethylammonium bromide, benzyltrimethylammonium chloride, benzyltriethylammonium chloride, benzyltrimethylammonium hydroxide, benzyltriethylammonium hydroxide, benzyltributylammonium chloride, benzyltributylammonium bromide, tetrabutylphosphonium bromide, tetrabutylphosphonium chloride, tributylhexadecylphosphonium bromide, butyltriphenylphosphonium chloride, ethyltrioctylphosphonium bromide, tetraphenylphosphonium bromide.

The processes according to the invention for preparing compounds of the general formula (I) are preferably carried out with the use of diluents. Diluents suitable for carrying out processes (a) and (b) according to the invention are, besides water, especially inert organic solvents. These include, in particular, aliphatic, alicyclic or aromatic, optionally halogenated hydrocarbons, such as, for example, benzine, benzene, toluene, xylene, chlorobenzene, dichlorobenzene, petroleum ether, hexane, cyclohexane, dichloromethane, chloroform, tetrachloromethane; ethers such as diethyl ether, diisopropyl ether, dioxane, tetrahydrofuran or ethylene glycol dimethyl or diethyl ether; ketones such as acetone, butanone or methyl isobutyl ketone; nitriles such as acetonitrile, propionitrile or butyronitrile; amides such as N,N-dimethyl-formamide N,N-dimethylacetamide, N-methylformanilide, N-methylpyrrolidone or hexamethylphosphoric triamide; esters such as methyl acetate or ethyl acetate, sulphoxides such as dimethyl sulphoxide, alcohols such as methanol, ethanol, n- or i-propanol, ethylene glycol monomethyl ether, ethylene glycol monoethyl ether, diethylene glycol monomethyl ether, diethylene glycol monoethyl ether, mixtures thereof with water or pure water.

The reaction temperatures for carrying out processes (a) and (b) according to the invention can be varied within a relatively wide range. The temperatures used are generally between 0° C. and 150° C., preferably between 10° C. and 120° C.

The processes according to the invention are generally carried out under atmospheric pressure. However, it is also possible to carry out the processes according to the invention under elevated or reduced pressure—in general between 0.1 bar and 10 bar.

The starting materials for carrying out the processes according to the invention are generally employed in approximately equimolar amounts. However, it is also possible to use one of the components in a relatively large excess. The reaction is generally carried out in a suitable diluent in the presence of a reaction auxiliary, and the reaction mixture is generally stirred at the required temperature for several hours. The working up is carried out by conventional methods (compare the Preparation Examples).

The active substances according to the invention can be used as defoliants, desiccants, herbicides and, in particular, as weedkillers. Weeds mean in the widest sense all plants which grow in places where they are unwanted. Whether the substances according to the invention act as total or selective herbicides depends essentially on the amount applied.

The active substances according to the invention can be used, for example, for the following plants:

Dicotyledonous weeds of the genera: Abutilon, Amaranthus, Ambrosia, Anoda, Anthemis, Aphanes, Atriplex, Bellis, Bidens, Capsella, Carduus, Cassia, Centaurea, Chenopodium, Cirsium, Convolvulus, Datura, Desmodium, Emex, Erysimum, Euphorbia, Galeopsis, Galinsoga, Galium, Hibiscus, Ipomoea, Kochia, Lamium, Lepidium, Lindemia, Matricaria, Mentha, Mercurialis, Mullugo, Myosotis, Papaver, Pharbitis, Plantago, Polygonum, Portulaca, Ranunculus, Raphanus, Rorippa, Rotala, Rumex, Salsola, Senecio, Sesbania, Sida, Sinapis, Solanum, Sonchus, Sphenoclea, Stellaria, Taraxacum, Thlaspi, Trifolium, Urtica, Veronica, Viola, Xanthium.

Dicotyledonous crops of the genera: Arachis, Beta, Brassica, Cucumis, Cucurbita, Helianthus, Daucus, Glycine, Gossypium, Ipomoea, Lactuca, Linum, Lycopersicon, Nicotiana, Phaseolus, Pisum, Solanum, Vicia.

Monocotyledonous weeds of the genera: Aegilops, Agropyron, Agrostis, Alopecurus, Apera, Avena, Brachiaria, Bromus, Cenchrus, Commelina, Cynodon, Cyperus, Dactyloctenium, Digitaria, Echinochloa, Eleocharis, Eleusine, Eragrostis, Eriochloa, Festuca, Fimbristylis, Heteranthera, Imperata, Ischaemum, Leptochloa, Lolium, Monochoria, Panicum, Paspalum, Phalaris, Phleum, Poa, Rottboellia, Sagittaria, Scirpus, Setaria, Sorghum.

Monocotyledonous crops of the genera: Allium, Ananas, Asparagus, Avena, Hordeum, Oryza, Panicum, Saccharum, Secale, Sorghum, Triticale, Triticum, Zea.

The use of the active substances according to the invention is, however, by no means confined to these genera but also extends in the same way to other plants.

Depending on the concentration, the active substances according to the invention are suitable for total weed control, for example on industrial sites and rail tracks and on paths and areas with and without tree growth. The active substances according to the invention can equally be used for controlling weeds in perennial crops, for example forests, ornamental tree plantings, orchards, vineyards, citrus groves, nut orchards, banana plantations, coffee plantations, tea plantations, rubber plantations, oil palm plantations, cocoa plantations, soft fruit plantings and hopfields, lawns and turf and pastures and for selective weed control in annual crops.

The compounds of the formula (I) according to the invention show strong herbicidal activity and a broad spectrum of action when used on the soil and on epigeal parts of plants. They are also suitable to a certain extent for selective control of monocotyledonous and dicotolyedonous crops, both by the pre-emergence and by the post-emergence method.

The active substances according to the invention can be used in particular concentrations or application rates also for controlling animal pests and fungal or bacterial diseases of plants. They can, where appropriate, also be employed as intermediates or precursors for synthesizing other active substances.

It is possible according to the invention to treat all plants and parts of plants. Plants mean in this connection all plants and plant populations such as desired and undesired wild plants or crop plants (including naturally occurring crop plants). Crop plants may be plants which can be obtained by conventional breeding and optimization methods or by biotechnological and genetic engineering methods or combinations of these methods, including transgenic plants and including plant varieties which can or cannot be protected by plant variety rights. Parts of plants mean all epigeal and hypogeal parts and organs of plants such as shoot, leaf, flower and root, listing by way of example the leaves, needles, stems, trunks, flowers, fruiting bodies, fruits and seeds, and roots, tubers and rhizomes. Parts of plants include also harvested materials, and vegetative and generative propagation material, for example cuttings, tubers, rhizomes, layers and seeds.

As already mentioned above, it is preferable according to the invention to treat all plants and parts thereof. In a preferred embodiment there is treatment of plants which grow wild (weeds, harmful plants) and/or plant species and plant varieties obtained by conventional biological breeding methods such as, for example, crossing or protoplast fusion (crop plants), and parts thereof. In another preferred embodiment there is treatment of transgenic plants and plant varieties obtained by genetic engineering methods, where appropriate in combination with conventional methods (genetically modified organisms) and parts thereof. The term “parts” or “parts of plants” has been explained above.

Plant varieties mean crop plants with particular properties (“traits”) which have been obtained by conventional breeding, by mutagenesis or by recombinant DNA techniques. These may be varieties, biotypes and genotypes.

The transgenic plants or plant varieties (obtained by genetic engineering) to be treated according to the invention include all plants which, through the genetic modification, have received genetic material which confers on these plants particular advantageous valuable properties (“traits”). Examples of such properties are better plant growth, increased tolerance of high or low temperatures, increased tolerance of aridity or of water or soil salinity, increased flowering performance, facilitated harvesting, accelerated maturation, higher yields, higher quality and/or higher nutritional value of the harvested products, better storability and/or processability of the harvested products. Other examples which are particularly emphasized of such properties are increased resistance of the plants to animal and microbial pests such as to insects, mites, phytopathogenic fungi, bacteria and/or viruses, and an increased tolerance of the plants to certain herbicidal active substances. Examples of transgenic plants which are mentioned are the important crop plants such as cereals (including rice), maize, soya, potato, cotton, beets, oilseed rape, cultivated grasses such as golf turf and lawns, fruit plants (with the fruits apples, pears, citrus fruits and grapes) and plantation crops such as oil and rubber trees, with particular emphasis on cereals (including rice), maize, soya, potato, cotton, beets and oilseed rape. Properties (“traits”) which are particularly emphasized are increased resistance of the plants to insects due to toxins produced in the plants, in particular those which are generated by the genetic material from Bacillus thuringiensis (for example through the gene CryIA(a), CryIA(b), CryIA(c), CryIIA, CryIIIA, CryIIIB2, Cry9c Cry2Ab, Cry3Bb and CryIF, and combinations thereof) in the plants (“Bt plants” hereinafter). Properties (“traits”) which are also particularly emphasized are increased resistance of plants to fungi, bacteria and viruses due to systemic acquired resistance (SAR), systemin, phytoalexins, elicitors, and resistance genes and correspondingly expressed proteins and toxins. Properties (“traits”) which are further particularly emphasized are increased tolerance of the plants toward particular herbicidal active substances and classes of active substance, such as glyphosate or glufosinate/phosphinothricin (for example “PAT” gene), ALS inhibitors such as imidazolinones, sulphonylureas and others, PPO inhibitors (for example plants with acuron genes), 4-HPD inhibitors such as isoxazoles (for example isoxaflutol), ACCase inhibitors such as sethoxydim, and bromoxynil.

The genes conferring the required properties (“traits”) in each case may also occur in combinations with one another in the transgenic plants. Examples which may be mentioned of “Bt plants” are maize varieties, cotton varieties, soya varieties and potato varieties, which are marketed under the proprietary name YIELD GARD® (for example maize, cotton, soya), KnockOut® (for example maize), StarLinkg (for example maize), Bollgard® (cotton), Nucoton® (cotton) and NewLeafe (potato). Examples of herbicide-tolerant plants which may be mentioned are maize varieties, cotton varieties, soya varieties, cereal varieties including rise varieties, beet varieties and oilseed rape varieties, which are marketed under the proprietary names Roundup Ready® (glyphosate tolerance, for example maize, cotton, soya, beets, oilseed rape), Liberty Link® (glufosinate tolerance, for example oilseed rape, maize, beets), IMI® (imidazolinone tolerance) and STS® (sulphonylurea tolerance, for example maize). Mention may also be made of the varieties marketed under the name Clearfield® (for example maize, rice) as herbicide-resistant plants (conventionally bred for herbicide tolerance).

These statements also, of course, apply to plant varieties which will be developed in the future or come on the market in the future having the genetic properties (“traits”) or traits to be developed in the future. Depending on the plant species and plant varieties, their location and growth conditions (soils, climate, vegetation period, nutrition), the treatment according to the invention may also lead to superadditive (“synergistic”) effects. Thus, for example, reduced application rates and/or extensions of the range of action and/or an enhancement of the effect of the substances and compositions which can be used according to the invention—also in combination with other agrochemical active substances-, improved plant growth of the crop plants, increased tolerance of the crop plants towards high or low temperatures, increased tolerance of the crop plants to aridity or to water or soil salinity, increased flowering performance, facilitated harvesting, accelerated maturation, higher yields, higher quality and/or higher nutritional value of the harvested products, better storability and/or processability of the harvested products are possible and exceed the effects actually to be expected.

The plants mentioned can be treated particularly advantageously according to the invention with the compounds of the general formula I or the active substance mixtures according to the invention, with a good control of weeds being supplemented by the abovementioned synergistic effects with the plants or plant varieties described above. The preferred ranges indicated above for the active substances or mixtures also apply to the treatment of these plants. Treatment of plants with the compounds or mixtures specifically mentioned in the present text may be particularly emphasized.

The treatment according to the invention of the plants and parts of plants with the active substances takes place directly or through action on their environment, habitat or storage space by conventional treatment methods, for example by dipping, spraying, vaporizing, atomizing, broadcasting, brushing on and, in the case of propagation material, in particular in the case of seeds, also by monolayer or multilayer coatings.

The active substances can be converted into conventional formulations such as solutions, emulsions, wettable powders, suspensions, powders, dusts, pastes, soluble powders, granules, suspension/emulsion concentrates, active substance-impregnated natural or synthetic substances, and microfine encapsulations in polymeric substances.

These formulations are produced in a known manner, for example by mixing the active substances with extenders, that is to say liquid solvents and/or solid carriers, where appropriate with use of surfactants, that is to say emulsifiers and/or dispersants and/or foam-forming agents.

In the case of the use of water as extender it is also possible, for example, to use organic solvents as cosolvents. Suitable liquid solvents are essentially: aromatic compounds such as xylene, toluene, or alkylnaphthalenes, chlorinated aromatic compounds and chlorinated aliphatic hydrocarbons, such as chlorobenzenes, chloroethylenes or methylene chloride, aliphatic hydrocarbons such as cyclohexane or paraffins, for example petroleum fractions, mineral and vegetable oils, alcohols such as butanol or glycol, and their ethers and esters, ketones such as acetone, methyl ethyl ketone, methyl isobutyl ketone or cyclohexanone, highly polar solvents such as dimethylformamide and dimethyl sulphoxide, and water.

Suitable solid carriers are: for example ammonium salts and ground natural minerals such as kaolins, aluminas, talc, chalk, quartz, attapulgite, montmorillonite or diatomaceous earth and ground synthetic minerals such as highly disperse silica, aluminium oxide and silicates, suitable solid carriers for granules are: for example crushed and fractionated natural minerals such as calcite, marble, pumice, sepiolite, dolomite and synthetic granules from ground inorganic and organic materials, and granules from organic material such as sawdust, coconut shells, maize cobs and tobacco stalks; suitable emulsifiers and/or foam-forming agents are: for example nonionic and anionic emulsifiers such as polyoxyethylene fatty acid esters, polyoxyethylene fatty alcohol ethers, for example alkylaryl polyglycol ether, alkyl-sulphonates alkyl sulphates, arylsulphonates and protein hydrolysates; suitable dispersants are: for example lignin sulphite waste liquors and methylcellulose.

Adhesives such as carboxymethylcellulose, natural and synthetic polymers in the form of powders, granules or latices, such as gum arabic, polyvinyl alcohol, polyvinyl acetate, and natural phospholipids such as cephalins and lecithins, and synthetic phospholipids, can be used in the formulations. Other additives may be mineral and vegetable oils.

It is possible to use colorants such as inorganic pigments, for example iron oxide, titanium oxide, Prussian blue and organic colorants such as alizarin, azo and metal phthalocyanine dyes and trace nutrients such as salts of iron, manganese, boron, copper, cobalt, molybdenum and zinc.

The formulations generally contain between 0.1 and 95 per cent by weight of active substance, preferably between 0.5 and 90%.

The active substances according to the invention can be used as such or in their formulations, also mixed with known herbicides and/or with substances which improve the crop plant tolerance (“safeners”) for controlling weeds, possibilities being ready-to-use formulations or tank mixtures. Mixtures with weed control compositions which contain one or more known herbicides and a safener are also possible.

Known herbicides are suitable for the mixtures, for example acetochlor, acifluorfen (-sodium), aclonifen, alachlor, alloxydim (-sodium), ametryne, amicarbazone, amidochlor, amidosulfuron, anilofos, asulam, atrazine, azafenidin, azimsulfuron, beflubutamid, benazolin (-ethyl), benfuresate, bensulfuron (-methyl), bentazon, benzfendizone, benzobicyclon, benzofenap, benzoylprop (-ethyl), bialaphos, bifenox, bispyribac (-sodium), bromobutide, bromofenoxim, bromoxynil, butachlor, butafenacil (-allyl), butroxydim, butylate, cafenstrole, caloxydim, carbetamide, carfentrazone (-ethyl), chlomethoxyfen, chloramben, chloridazon, chlorimuron (-ethyl), chlornitrofen, chlorsulfuron, chlortoluron, cinidon (-ethyl), cinmethylin, cinosulfuron, clefoxydim, clethodim, clodinafop (-propargyl), clomazone, clomeprop, clopyralid, clopyrasulfuron (-methyl), cloransulam (-methyl), cumyluron, cyanazine, cybutryne, cycloate, cyclosulfamuron, cycloxydim, cyhalofop (-butyl), 2,4-D, 2,4-DB, desmedipham, diallate, dicamba, dichlorprop (—P), diclofop (-methyl), diclosulam, diethatyl (-ethyl), difenzoquat, diflufenican, diflufenzopyr, dimefuron, dimepiperate, dimethachlor, dimethametryn, dimethenamid, dimexyflam, dinitramine, diphenamid, diquat, dithiopyr, diuron, dymron, epropodan, EPTC, esprocarb, ethalfluralin, ethametsulfuron (-methyl), ethofumesate, ethoxyfen, ethoxysulfuron, etobenzanid, fenoxaprop (—P-ethyl), fentrazamide, flamprop (-isopropyl, -isopropyl-L, -methyl), flazasulfuron, florasulam, fluazifop (—P-butyl), fluazolate, flucarbazone (-sodium), flufenacet, flumetsulam, flumiclorac (-pentyl), flumioxazin, flumipropyn, fluometuron, fluorochloridone, fluoroglycofen (-ethyl), flupoxam, flupropacil, flurpyrsulfuron (-methyl, -sodium), flurenol (-butyl), fluridone, fluroxypyr (-butoxypropyl, -meptyl), flurprimidol, flurtamone, fluthiacet (-methyl), fluthiamide, fomesafen, foramsulfuron, glufosinate (-ammonium), glyphosate (-isopropyl-ammonium), halosafen, haloxyfop (-ethoxyethyl, —P-methyl), hexazinone, imazamethabenz (-methyl), imazamethapyr, imazamox, imazapic, imazapyr, imazaquin, imazethapyr, imazosulfuron, iodosulfuron (-methyl, -sodium), ioxynil, isopropalin, isoproturon, isouron, isoxaben, isoxachlortole, isoxaflutole, isoxapyrifop, lactofen, lenacil, linuron, MCPA, mecoprop, mefenacet, mesotrione, metamitron, metazachlor, methabenzthiazuron, metobenzuron, metobromuron, (alpha-) metolachlor, metosulam, metoxuron, metribuzin, metsulfuron (-methyl), molinate, monolinuron, naproanilide, napropamide, neburon, nicosulfuron, norflurazon, orbencarb, oryzalin, oxadiargyl, oxadiazon, oxasulfuron, oxaziclomefone, oxyfluorfen, paraquat, pelargonic acid, pendimethalin, pendralin, pentoxazone, phenmedipham, picolinafen, piperophos, pretilachlor, primisulfuron (-methyl), profluazol, prometryn, propachlor, propanil, propaquizafop, propisochlor, propoxycarbazone (-sodium), propyzamide, prosulfocarb, prosulfuron, pyraflufen (-ethyl), pyrazogyl, pyrazolate, pyrazosulfuron (-ethyl), pyrazoxyfen, pyribenzoxim, pyributicarb, pyridate, pyridatol, pyriftalid, pyriminobac (-methyl), pyrithiobac (-sodium), quinchlorac, quinmerac, quinoclamine, quizalofop (—P-ethyl, —P-tefuryl), rimsulfuron, sethoxydim, simazine, simetryn, sulcotrione, sulfentrazone, sulfometuron (-methyl), sulfosate, sulfosulfuron, tebutam, tebuthiuron, tepraloxydim, terbuthylazine, terbutryn, thenylchlor, thiafluamide, thiazopyr, thidiazimin, thifensulfuron (-methyl), thiobencarb, tiocarbazil, tralkoxydim, triallate, triasulfuron, tribenuron (-methyl), triclopyr, tridiphane, trifluralin, trifloxysulfuron, triflusulfuron (-methyl), tritosulfuron.

A mixture with other known active substances such as fungicides, insecticides, acaricides, nematicides, substances to protect against bird feeding, plant nutrients and soil conditioners is also possible.

The active substances can be applied as such, in the form of their formulations or of the use forms prepared therefrom by further dilution, such as ready-to-use solutions, suspensions, emulsions, powders, pastes and granules. Application takes place in a conventional way, for example by pouring, spraying, atomizing, broadcasting.

The active substances according to the invention can be applied both before and after emergence of the plants. They can also be incorporated into the soil before sowing.

The active substance application rate may vary within a relatively wide range. It depends essentially on the nature of the desired effect. The application rates are in general between 1 g and 10 kg of active substance per hectare of soil area, preferably between 5 g and 5 kg per ha.

The preparation and use of the active substances according to the invention is evident from the following examples.

### PREPARATION EXAMPLES

### Example 1

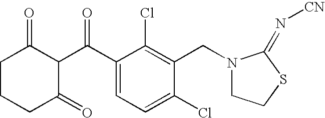

(Process (a))

A mixture of 1.7 g (5 mmol) of 2,4-dichloro-3-[(2-cyanoimino-1,3-thiazolidin-3-yl)-methyl]benzoic acid, 0.7 g (5 mmol) of cyclohexane-1,3-dione, 1.3 g (6 mmol) of dicyclohexylcarbodiimide and 100 ml of acetonitrile is stirred at room temperature (about 20° C.) for 60 hours. After addition of 1.5 g (15 mmol) of triethylamine and 0.9 g (10 mmol) of acetone cyanohydrin, the reaction mixture is stirred at room temperature for a further 20 hours and then concentrated under reduced pressure. The residue is taken up in methylene chloride and washed with water. The organic phase is then mixed with 100 ml of water and adjusted to pH=11 by adding potassium carbonate. The aqueous phase is separated off and mixed with 100 ml of methylene chloride. It is then acidified with IN hydrochloric acid, and the organic phase is separated off, washed with water and with saturated aqueous sodium chloride solution, dried with sodium sulphate and filtered. The filtrate is concentrated under reduced pressure, the residue is digested with diethyl ether/isopropanol, and the resulting crystalline product is isolated by filtration with suction.

0.5 g (24% of theory) of 3-[2,6-dichloro-3-[(2,6-dioxocyclohexyl)carbonyl]benzyl]-1,3-thiazolidin-2-ylidenecyanamide of melting point 1 54° C. is obtained.

### Example 2

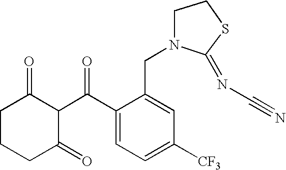

(Process (a))

A mixture of 4.0 g (12.2 mmol) of 2-[(2-cyanoimino-1,3-thiazolidin-3-yl)methyl]-4-trifluoromethylbenzoic acid, 1.36 g (12.2 mmol) of cyclohexane-1,3-dione, 3.0 g (18 mmol) of dicyclohexylcarbodiimide and 100 ml of acetonitrile is stirred at, room temperature (about 20° C.) for 20 hours. After addition of 3.4 ml (24.4 mmol) of triethylamine and 0.65 ml (12 mmol) of trimethylsilyl cyanide, the reaction mixture is stirred at room temperature for a further 2 hours and concentrated under reduced pressure. The residue is taken up in 10% strength aqueous sodium carbonate solution and filtered. The filtrate is shaken with diethyl ether, and the aqueous phase is then acidified with 2N hydrochloric acid and subsequently shaken with methylene chloride. The methylene chloride phase is dried with sodium sulphate and filtered. The filtrate is concentrated under reduced pressure, the residue is crystallized from diethyl ether/petroleum ether, and the product is isolated by filtration with suction.

3.76 g (73% of theory) of 3-[2-[(2,6-dioxocyclohexyl)carbonyl]-5-trifluoromethyl-benzyl]-1,3-thiazolidin-2-ylidenecyanamide are obtained.

LogP (at pH=2.3): 2.54.

It is possible in analogy to Examples 1 and 2 and in accordance with the general description of the preparation process according to the invention to prepare, for example, also the compounds of the general formula (I)—and of the formulae (I-1), (I-2) or (I-3)—which are listed in Table 1 below.

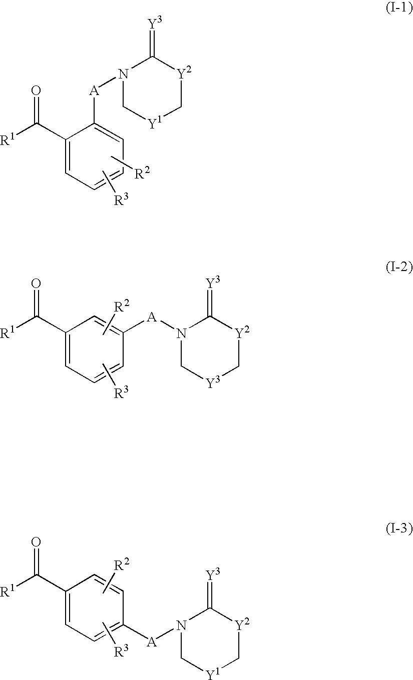

TABLE 1
Examples of compounds of the formula (I)

| Ex. | (Position) | | (Position) | (Position) | | | | Formula |
|---|---|---|---|---|---|---|---|---|
| No. | A | R¹ | R² | R³ | Y¹ | Y² | Y³ | Physical data |
| 3 | (3) CH₂ | 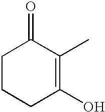 | (2) Cl | (4) Cl | — | N—CH₃ | N—CN | (I-2) |
| 4 | (3) CH₂ | 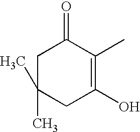 | (2) Cl | (4) Cl | — | N—CH₃ | N—CN | (I-2) |
| 5 | (3) CH₂ | 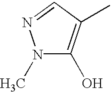 | (2) Cl | (4) Cl | — | N—CH₃ | N—CN | (I-2) |
| 6 | (3) CH₂ | 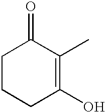 | (2) Cl | (4) Cl | CH₂ | N—CH₃ | N—CN | (I-2) |
| 7 | (3) CH₂ | 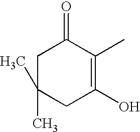 | (2) Cl | (4) Cl | CH₂ | N—CH₃ | N—CN | (I-2) |
| 8 | (3) CH₂ | 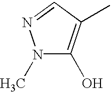 | (2) Cl | (4) Cl | CH₂ | N—CH₃ | N—CN | (I-2) |
| 9 | (3) CH₂ | 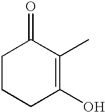 | (2) Cl | (4) OCH₃ | — | S | N—CN | (I-2) |
| 10 | (3) CH₂ | 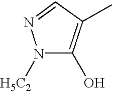 | (2) Cl | (4) Cl | — | S | N—CN | (I-2) m.p.: 148° C. |
| 11 | (3) CH₂ | 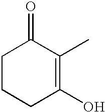 | (2) Cl | (4) CO₂CH₃ | — | S | N—CN | (I-2) m.p.: 208° C. |
| 12 | (3) CH₂ | 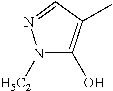 | (2) Cl | (4) SO₂CH₃ | — | S | N—CN | (I-2) m.p.: 190° C. |
| 13 | (2) CH₂ | 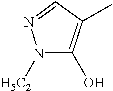 | — | (4) CF₃ | — | S | N—CN | (I-1) |
| 14 | (3) CH₂ | 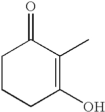 | (2) Cl | (4) Cl | — | N—C₂H₅ | N—CN | (I-2) logP =2.37^{a)} |
| 15 | (3) CH₂ | 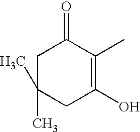 | (2) Cl | (4) Cl | — | N—C₂H₅ | (I-2) logP =2.92^{a)} | |
| 16 | (3) CH₂ | 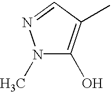 | (2) Cl | (4) Cl | — | N—C₂H₅ | N—CN | (I-2) |
| 17 | (3) CH₂ | 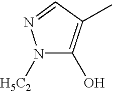 | (2) Cl | (4) Cl | — | N—C₂H₅ | N—CN | (I-2) |
| 18 | (3) CH₂ | 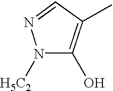 | (2) Cl | (4) Cl | — | N—CH₃ | N—CN | (I-2) |
| 19 | (3) CH₂ | 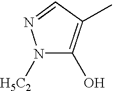 | (2) Cl | (4) Cl | CH₂ | N—CH₃ | N—CN | (I-2) |
| 20 | (4) CH₂ | 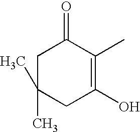 | (2) NO₂ | — | — | N—CH₃ | N—CN | (I-3)logP=2.40^{a)} |
| 21 | (4) CH₂ | 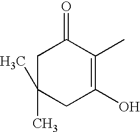 | (2) NO₂ | — | — | N—CH₃ | N—CN | (I-3) |
| 22 | (4) CH₂ | 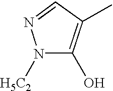 | (2) NO₂ | — | — | N—CH₃ | N—CN | (I-3) |
| 23 | (4) CH₂ | 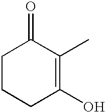 | (2) NO₂ | — | — | N—CH₃ | N—CN | (I-3) |
| 24 | (3) CH₂ | 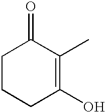 | (2) Cl | (4) Cl | — | N—C₃H₇-i | N—CN | (I-2) |
| 25 | (3) CH₂ | 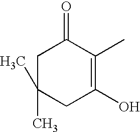 | (2) Cl | (4) Cl | — | N—C₃H₇-i | N—CN | (I-2) logP=3.16^{a)} |
| 26 | (2) CH₂ | 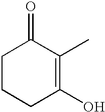 | — | (4) Br | — | S | N—CN | (I-1) logP =2.36^{a)} |
| 27 | (2) CH₂ | 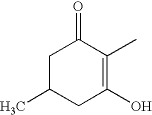 | — | (4) Br | — | S | N—CN | (I-1) logP =2.65^{a)} |
| 28 | (2) CH₂ | 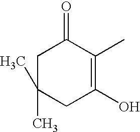 | — | (4) Br | — | S | N—CN | (I-1) logP =2.87^{a)} |
| 29 | (2) CH₂ | 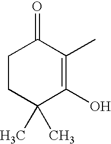 | — | (4) Br | — | S | N—CN | (I-1) logP =2.95^{a)} |
| 30 | (2) CH₂ | 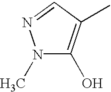 | — | (4) Br | — | S | N—CN | (I-1) logP =2.24^{a)} |
| 31 | (2) CH₂ | 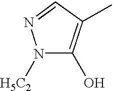 | — | (4) Br | — | S | N—CN | (I-1) logP =2.46^{a)} |
| 32 | (2) CH₂ | 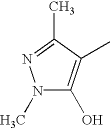 | — | (4) Br | — | S | N—CN | (I-1) logP =2.38^{a)} |
| 33 | (2) CH₂ | 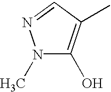 | — | (4) CF₃ | CH₂ | N—CH₃ | N—CN | (I-1) logP =1.68^{a)} |
| 34 | (2) CH₂ | 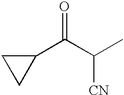 | — | (4) CN | — | S | N—CN | (I-1) logP =1.83^{a)} |
| 35 | (2) CH₂ | 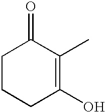 | — | (4) CN | — | S | N—CN | (I-1) logP =1.82^{a)} |
| 36 | (2) CH₂ | 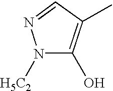 | — | (4) CN | — | S | N—CN | (I-1) |
| 37 | (2) CH₂ | 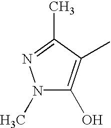 | — | (4) CN | — | S | N—CN | (I-1) logP =1.02^{a)} |

Determination of the logP values indicated in Table 1 took place in accordance with EEC Directive 79/831 Annex V.A8 by HPLC (high performance liquid chromatography) on a reversed phase column (C 18). Temperature: 43° C.

(a) Eluent for determination in the acidic range: 0.1% aqueous phosphoric acid, acetonitrile; linear gradient from 10% acetonitrile to 90% acetonitrile—corresponding measured results are marked with ^{a}) in Table 1.

(b) Eluent for determination in the neutral range: 0.01 molar aqueous phosphate buffer solution, acetonitrile; linear gradient from 10% acetonitrile to 90% acetonitrile—corresponding measured results are marked with ^{b}) in Table 1.

Calibration took place with unbranched alkan-2-ones (having 3 to 16 carbon atoms) whose logP values are known (determination of the logP values on the basis of the retention times by linear interpolation between two consecutive alkanones).

The lambda max values are determined on the basis of the UV spectra from 200 nm to 400 nm at the maxima of the chromatographic signals.

Starting Materials of the Formula (II):

### Example (II-1)

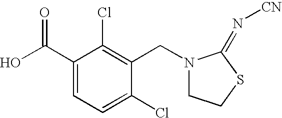

A mixture of 8.0 g (23 mmol) of methyl 2,4-dichloro-3-[(2-cyanoimino-1,3-thiazolidin-3-yl)methyl]benzoate, 0.9 g (22.5 mmol) of sodium hydroxide, 80 ml of 1,4-dioxane and 80 ml of water is heated to boiling for 5 minutes and then stirred at room temperature (about 20° C.) for 60 minutes. It is subsequently concentrated to about half the volume under reduced pressure, then diluted with 100 ml of water and acidified with 2N hydrochloric acid while stirring vigorously, and the resulting crystalline product is isolated by filtration with suction.

5.0 g (66% of theory) of 2,4-dichloro-3-[(2-cyanoimino-1,3-thiazolidin-3-yl)-methyl]benzoic acid of melting point 208° C. are obtained.

### Example (II-2)

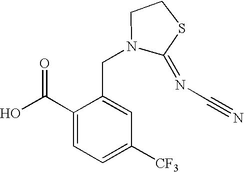

A mixture of 1.0 g (2.9 mmol) of methyl 2-[(2-cyanoimino-1,3-thiazolidin-3-yl)-methyl]-4-trifluoromethylbenzoate, 0.17 g (3.7 mmol) of potassium hydroxide, 5 ml of 2-methoxyethanol and 20 ml of cyclohexane is stirred at room temperature (about 20° C.) for 2 hours and then concentrated under reduced pressure. The residue is taken up in water and acidified with 2N hydrochloric acid, and the resulting crystalline product is isolated by filtration with suction.

0.76 g (80% of theory) of 2-[(2-cyanoimino-1,3-thiazolidin-3-yl)methyl]-4-trifluoro-methylbenzoic acid is obtained.

LogP (at pH=2.3): 2.01.

In analogy to Examples (11-1) and (11-2) it is possible, for example, also to prepare the compounds of the general formula (II)—or of the general formula (IIb)—which are listed in Table 2 below.

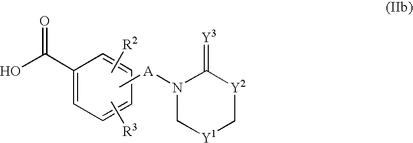

TABLE 2
Examples of compounds of the formula (IIb)

| Ex. | | (Position) | (Position) | | | | Physical |
|---|---|---|---|---|---|---|---|
| No. | (Position) A | R² | R³ | Y¹ | Y² | Y³ | data |
| II-3 | (3) | (2) | (4) | — | S | N—CN | m.p.: 196° C. |
| | CH₂ | Cl | SO₂CH₃ | | | | |
| II-4 | (3) | (2) | (4) | — | N—CH₃ | N—CN | |
| | CH₂ | Cl | Cl | | | | |
| II-5 | (3) | (2) | (4) | CH₂ | N—CH₃ | N—CN | |
| | CH₂ | Cl | Cl | | | | |
| II-6 | (3) | (2) | (4) | — | N—C₂H₅ | N—CN | |
| | CH₂ | Cl | Cl | | | | |
| II-7 | (3) | (2) | (4) | — | S | N—CN | |
| | CH₂ | OCH₃ | Cl | | | | |

Starting Materials of the Formula (IIa):

### Example (IIa-1)

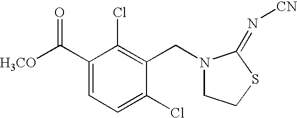

7.0 g (55 mmol) of 1,3-thiazolidin-2-ylidenecyanamide are introduced into 100 ml of tetrahydrofuran and, after addition of 1.4 g (55 mmol) of sodium hydride (95% pure), stirred at room temperature (about 20° C.) until gas evolution ceases. Then a solution of 15 g (50 mmol) of methyl 3-bromomethyl-2,4-dichlorobenzoate in 40 ml of tetrahydrofuran is added dropwise thereto, and the reaction mixture is stirred at room temperature for 2 hours. It is then concentrated under reduced pressure the residue is taken up in methylene chloride, washed with weakly acidic water and saturated aqueous sodium chloride solution, dried with sodium sulphate and filtered. The filtrate is concentrated under reduced pressure, the residue is digested with diethyl ether, and the crystalline product is isolated by filtration with suction.

14.4 g (76% of theory) of methyl 2,4-dichloro-3-[(2-cyanoimino-1,3-thiazolidin-3-yl)-methyl]benzoate of melting point 158° C. are obtained.

### Example (IIa-2)

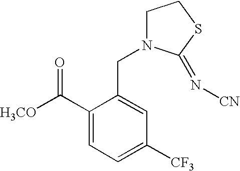

A mixture of 20.9 g (70 mmol) of methyl 2-bromomethyl-4-trifluoromethylbenzoate, 8.95 g (70 mmol) of 1,3-thiazolidin-2-ylidenecyanamide, 19 g (140 mmol) of potassium carbonate, 50 ml of acetone and 200 ml of acetonitrile is heated to boiling under reflux for 4 hours and then concentrated under reduced pressure. The residue is taken up in water and shaken with methylene chloride. The organic phase is dried with sodium sulphate and filtered. The filtrate is concentrated under reduced pressure, the residue is digested with petroleum ether, and the crystalline product is isolated by filtration with suction.

18 g (74.5% of theory) of methyl 2-[(2-cyanoimino-1,3-thiazolidin-3-yl)methyl]-4-trifluoromethylbenzoate are obtained.

LogP (at pH=2.3): 2.53.

It is possible in analogy to Examples (IIa-1) and (IIa-2) for example also to prepare the compounds of the general formula (IIa)—or of the general formula (IIc)—which are listed in Table 3 below.

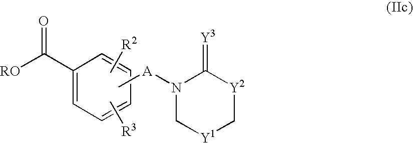

TABLE 3
Examples of compounds of the formula (IIc)

| Ex. | | | (Position) | (Position) | | | | Physical |
|---|---|---|---|---|---|---|---|---|
| No. | (Position) A | R | R² | R³ | Y¹ | Y² | Y³ | data |
| IIa-3 | (3) | CH₃ | (2) | (4) | — | S | N—CN | m.p.: |
| | CH₂ | | Cl | SO₂CH₃ | | | | 190° C. |
| IIa-4 | (3) | CH₃ | (2) | (4) | — | N—CH₃ | N—CH₃ | |
| | CH₂ | | Cl | Cl | | | | |
| IIa-5 | (3) | CH₃ | (2) | (4) | CH₂ | N—CH₃ | N—CN | |
| | CH₂ | | Cl | Cl | | | | |
| IIa-6 | (3) | CH₃ | (2) | (4) | — | N—C₂H₅ | N—CN | |
| | CH₂ | | Cl | Cl | | | | |
| IIa-7 | (4) | CH₃ | (2) | — | — | N—CH₃ | N—CN | |
| | CH₂ | | NO₂ | | | | | |
| IIa-8 | (3) | CH₃ | (2) | (4) | — | S | N—CN | |
| | CH₂ | | OCH₃ | Cl | | | | |
| IIa-9 | (2) | CH₃ | (4) | — | CH₂ | N—CH₃ | N—CN | logP = 2.20^{a)} |
| | CH₂ | | CF₃ | | | | | |

### Use Examples

### Example A

Pre-emergence test

| | |
|---|---|
| Solvent: | 5 parts by weight of acetone |
| Emulsifier: | 1 part by weight of alkylaryl polyglycol ether |

To produce an expedient preparation of active substance, 1 part by weight of active substance is mixed with the stated amount of solvent, the stated amount of emulsifier is added and the concentrate is diluted with water to the required concentration.

Seeds of the test plants are sown in normal soil. After 24 hours, the soil is sprayed with the active substance preparation in such a way that the respective desired application rate of active substance per unit area is achieved. The active substance concentration in the spray liquor is selected so that the respective required application rate of active substance is achieved with 1000 litres of water per hectare.

After three weeks, the degree of damage to the plants is assessed in % damage by comparison with the development of the untreated control. The meanings are:

| | |
|---|---|
| 0% = | no effect (as untreated control) |
| 100% = | total destruction |

In this test, for example, the compounds of Preparation Example 1, 2, 10, 11 and 13 show a strong effect on weeds while, in some cases, being well tolerated by crop plants such as, for example, maize.

### Example B

Post-Emergence Test

| | |
|---|---|
| Solvent: | 5 parts by weight of acetone |
| Emulsifier: | 1 part by weight of alkylaryl polyglycol ether |

To produce an expedient preparation of active substance, 1 part by weight of active substance is mixed with the stated amount of solvent, the stated amount of emulsifier is added, and the concentrate is diluted with water to the required concentration.

The active substance preparation is used to spray test plants which are 5-15 cm high in such a way that the respective desired application rate of active substance per unit area is achieved. The concentration of the spray liquor is chosen so that the respective required application rates of active substance are achieved in 1000 l of water/ha.

After three weeks, the degree of damage to the plants is assessed in % damage by comparison with the development of the untreated control. The meanings are:

0%=no effect (as untreated control)

100%=total destruction

In this test, for example, the compounds of Preparation Example 1, 10 and 11 show a strong effect on weeds while, in some cases, being well tolerated by crop plants such as, for example, maize and wheat.

1. Compounds of the formula (I) 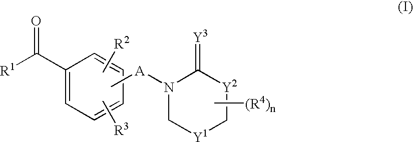
in which
n represents the numbers 0, 1, 2, 3 or 4,
A represents alkanediyl (alkylene),
R¹ represents one of the following groups 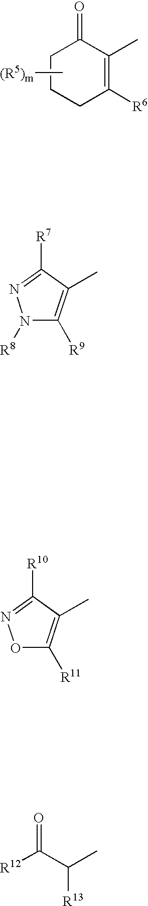
where
m represents the numbers 0 to 6,
R⁵ represents halogen or represents in each case optionally substituted alkyl, alkylthio or aryl or—in the case where m represents 2—optionally also together with a second R⁵ radical represents oxygen or alkanediyl (alkylene),
R⁶ represents hydroxyl, formyloxy, halogen, or represents in each case optionally substituted alkoxy, alkylthio, alkylsulphinyl, alkylsulphonyl, alkylcarbonyloxy, alkoxycarbonyloxy, alkylaminocarbonyloxy, alkylsulphonyloxy, alkenyloxy, alkinyloxy, aryloxy, arylthio, arylsulphinyl, arylsulphonyl, arylcarbonyloxy, arylcarbonylalkoxy, arylsulphonyloxy, arylalkoxy, arylalkylthio, arylalkylsulphinyl or arylalkylsulphonyl,
R⁷ represents hydrogen, cyano, carbamoyl, thiocarbamoyl, halogen, or represents in each case optionally substituted alkyl, alkoxy, alkylthio, alkylsulphinyl, alkylsulphonyl, alkoxycarbonyl or cycloalkyl,
R⁸ represents hydrogen or represents in each case optionally substituted alkyl, alkenyl, alkinyl, cycloalkyl, cycloalkylalkyl, aryl or arylalkyl,
R⁹ represents hydroxyl, formyloxy or represents in each case optionally substituted alkoxy, alkylcarbonyloxy, alkoxycarbonyloxy, alkylaminocarbonyloxy, alkylsulphonyloxy, alkenyloxy, alkinyloxy, arylalkoxy, arylcarbonyloxy, arylcarbonylalkoxy or arylsulphonyloxy,
R¹⁰ represents hydrogen, cyano, carbamoyl, thiocarbamoyl, halogen or represents in each case optionally substituted alkyl, alkylcarbonyl, alkoxy, alkoxycarbonyl, alkylthio, alkylsulphinyl or alkylsulphonyl,
R¹¹ represents hydrogen or represents in each case optionally substituted alkyl or cycloalkyl,
R¹² represents hydrogen or represents in each case optionally substituted alkyl or cycloalkyl, and
R¹³ represents hydrogen, cyano, carbamoyl, halogen, or represents in each case optionally substituted alkyl, alkoxy, alkoxycarbonyl, alkylthio, alkylsulphinyl or alkylsulphonyl,
R² represents hydrogen, nitro, cyano, carboxyl, carbamoyl, thiocarbamoyl, halogen or represents in each case optionally substituted alkyl, alkoxy, alkylthio, alkylsulphinyl, alkylsulphonyl, alkylamino, dialkylamino or dialkylaminosulphonyl,
R³ represents hydrogen, nitro, cyano, carboxyl, carbamoyl, thiocarbamoyl, halogen or represents in each case optionally substituted alkyl, alkoxy, alkylthio, alkylsulphinyl, alkylsulphonyl, alkylamino, dialkylamino or dialkylaminosulphonyl, and
R⁴ represents optionally substituted alkyl,
Y¹ represents a single bond, O (oxygen), S (sulphur), N—Z or optionally substituted (by R⁴) alkanediyl,
Y² represents S (sulphur) or N—Z,
Y³ represents N—Y⁴ or C(Y⁴,Y⁵) or—in the case where Y¹ represents O (oxygen), S (sulphur) or N—Z—also represents O (oxygen) or S (sulphur),
Y⁴ represents cyano, nitro or represents in each case optionally substituted alkylcarbonyl, alkylsulphonyl, arylcarbonyl or arylsulphonyl,
Y⁵ represents hydrogen, cyano, nitro or represents in each case optionally substituted alkylcarbonyl, alkylsulphonyl, arylcarbonyl or arylsulphonyl,
Z represents hydrogen or represents in each case optionally substituted alkyl, alkenyl or alkinyl,
including all possible tautomeric and/or stereoisomeric forms of the compounds of the general formula (I) and the possible salts and acid or base adducts of the compounds of the general formula (1).

2. Compounds of the formula (I) according to claim 1, characterized in that
n represents the numbers 0, 1, 2 or 3,
A represents alkanediyl (alkylene) having 1 to 6 carbon atoms,
R² represents hydrogen, nitro, cyano, carboxyl, carbamoyl, thiocarbamoyl, halogen or represents in each case optionally cyano-, halogen-, C₁-C₄-alkoxy-, C₁-C₄-alkylthio-, C₁-C₄-alkylsulphinyl- or C₁-C₄-alkylsulphonyl-substituted alkyl, alkoxy, alkylthio, alkylsulphinyl, alkylsulphonyl, alkylamino, dialkylamino or dialkylaminosulphonyl having, in each case, 1 to 6 carbon atoms in the alkyl groups,
R³ represents hydrogen, nitro, cyano, carboxyl, carbamoyl, thiocarbamoyl, halogen or represents in each case optionally cyano-, halogen-, C₁-C₄-alkoxy-, C₁-C₄-alkylthio-, C₁-C₄-alkylsulphinyl- or C₁-C₄-alkylsulphonyl-substituted alkyl, alkoxy, alkylthio, alkylsulphinyl, alkylsulphonyl, alkylamino, dialkylamino or dialkylaminosulphonyl having, in each case, 1 to 6 carbon atoms in the alkyl groups,
R⁴ represents optionally cyano- or halogen-substituted alkyl having 1 to 6 carbon atoms,
m represents the numbers 0 to 4,
R⁵ represents halogen, represents in each case optionally halogen-substituted alkyl or alkylthio having, in each case, 1 to 6 carbon atoms or represents phenyl or—in the case where m represents 2—optionally also together with a second R⁵ radical represents oxygen or alkanediyl (alkylene) having 3 to 5 carbon atoms,
R⁶ represents hydroxyl, formyloxy, halogen, represents in each case optionally halogen-substituted alkoxy, alkylthio, alkylsulphinyl, alkylsulphonyl, alkylcarbonyloxy, alkoxycarbonyloxy, alkylaminocarbonyloxy or alkylsulphonyloxy having, in each case, 1 to 6 carbon atoms in the alkyl groups, represents in each case optionally halogen-substituted alkenyloxy or alkinyloxy having, in each case, 3 to 6 carbon atoms, represents in each case optionally nitro-, cyano-, halogen-, C₁-C₄-alkyl-, C₁-C₄-halogenoalkyl-, C₁-C₄-alkoxy-C₁-C₄-halogenoalkoxy-, C₁-C₄-alkylthio-, C₁-C₄-halogenoalkylthio-, C₁-C₄-alkylsulphinyl-, C₁-C₄-halogenoalkylsulphinyl-, C₁-C₄-alkylsulphonyl- or C₁-C₄-halogenoalkylsulphonyl-substituted aryloxy, arylthio, arylsulphinyl, arylsulphonyl, arylcarbonyloxy, arylcarbonylalkoxy, arylsulphonyloxy, arylalkoxy, arylalkylthio, arylalkylsulphinyl or arylalkylsulphonyl having, in each case, 6 or 10 carbon atoms in the aryl group and, where appropriate, 1 to 4 carbon atoms in the alkyl moiety,
R⁷ represents hydrogen, cyano, carbamoyl, thiocarbamoyl, halogen, represents in each case optionally halogen-substituted alkyl, alkoxy, alkylthio, alkylsulphinyl, alkylsulphonyl, alkoxycarbonyl having, in each case, 1 to 6 carbon atoms in the alkyl groups, or represents optionally cyano-, halogen- or C₁-C₄-alkyl-substituted cycloalkyl having 3 to 6 carbon atoms,
R⁸ represents hydrogen, represents optionally cyano-, halogen- or C₁-C₄-alkoxy-substituted alkyl having 1 to 6 carbon atoms, represents in each case optionally cyano- or halogen-substituted alkenyl or alkinyl having, in each case, 3 to 6 carbon atoms, represents in each case optionally cyano-, halogen- or C₁-C₄-alkyl-substituted cycloalkyl or cycloalkylalkyl having, in each case, 3 to 6 carbon atoms in the cycloalkyl group and, where appropriate, 1 to 4 carbon atoms in the alkyl moiety, or represents in each case optionally nitro-, cyano-, halogen-, C₁-C₄-alkyl-, C₁-C₄-halogenoalkyl-, C₁-C₄-alkoxy-, C₁-C₄-halogenoalkoxy-, C₁-C₄-alkylthio-, C₁-C₄-halogenoalkylthio-, C₁-C₄-alkylsulphinyl-, C₁-C₄-halogenoalkylsulphinyl-, C₁-C₄-alkylsulphonyl- or C₁-C₄-halogenoalkylsulphonyl-substituted aryl or arylalkyl having, in each case, 6 or 10 carbon atoms in the aryl group and, where appropriate, 1 to 4 carbon atoms in the alkyl moiety,
R⁹ represents hydroxyl, formyloxy, represents in each case optionally cyano-, halogen- or C₁-C₄-alkoxy-substituted alkoxy, alkylcarbonyloxy, alkoxycarbonyloxy, alkylaminocarbonyloxy or alkylsulphonyloxy having, in each case, 1 to 6 carbon atoms in the alkyl groups, represents in each case optionally cyano- or halogen-substituted alkenyloxy or alkinyloxy having, in each case, 3 to 6 carbon atoms, or represents in each case optionally nitro-, cyano-, halogen-, C₁-C₄-alkyl-, C₁-C₄-halogenoalkyl-, C₁-C₄-alkoxy-, C₁-C₄-halogenoalkoxy-, C₁-C₄-alkylthio-, C₁-C₄-halogenoalkylthio-, C₁-C₄-alkylsulphinyl-, C₁-C₄-halogenoalkylsulphinyl-, C₁-C₄-alkylsulphonyl- or C₁-C₄-halogenoalkylsulphonyl-substituted arylalkoxy, arylcarbonyloxy, arylcarbonylalkoxy or arylsulphonyloxy having 6 or 10 carbon atoms in the aryl group and, where appropriate, 1 to 4 carbon atoms in the alkyl moiety,
R¹⁰ represents hydrogen, cyano, carbamoyl, thiocarbamoyl, halogen, or represents in each case optionally cyano-, halogen- or C₁-C₄-alkoxy-substituted alkyl, alkylcarbonyl, alkoxy, alkoxycarbonyl, alkylthio, alkylsulphinyl or alkylsulphonyl having, in each case, 1 to 6 carbon atoms in the alkyl groups,
R¹¹ represents hydrogen, represents optionally cyano-, halogen- or C₁-C₄-alkoxy-substituted alkyl having 1 to 6 carbon atoms or represents optionally cyano-, halogen- or C₁-C₄-alkyl-substituted cycloalkyl having 3 to 6 carbon atoms.
R¹² represents hydrogen, represents optionally cyano-, halogen- or C₁-C₄-alkoxy-substituted alkyl having 1 to 6 carbon atoms or represents optionally cyano-, halogen- or C₁-C₄-alkyl-substituted cycloalkyl having 3 to 6 carbon atoms,
R¹³ represents hydrogen, cyano, carbamoyl, halogen, or represents optionally in each case cyano-, halogen- or C₁-C₄-alkoxy-substituted alkyl, alkoxy, alkoxycarbonyl, alkylthio, alkylsulphinyl or alkylsulphonyl having, in each case, 1 to 6 carbon atoms in the alkyl groups,
Y¹ represents a single bond, O (oxygen), S (sulphur), N—Z or optionally substituted (by R⁴) alkanediyl having 1 to 4 carbon atoms,
Y⁴ represents cyano, nitro, represents in each case optionally halogen-substituted alkylcarbonyl or alkylsulphonyl having, in each case, up to 6 carbon atoms, or represents in each case optionally nitro-, cyano-, halogen-, C₁-C₄-alkyl-, C₁-C₄-halogenoalkyl-, C₁-C₄-alkoxy- or C₁-C₄-halogenoalkoxy-substituted arylcarbonyl or arylsulphonyl having, in each case, 6 or 10 carbon atoms,
Y⁵ represents hydrogen, cyano, nitro, represents in each case optionally halogen-substituted alkylcarbonyl or alkylsulphonyl having, in each case, up to 6 carbon atoms, or represents in each case optionally nitro-, cyano-, halogen-, C₁-C₄-alkyl-, C₁-C₄-halogenoalkyl-, C₁-C₄-alkoxy- or C₁-C₄-halogenoalkoxy-substituted arylcarbonyl or arylsulphonyl having, in each case, 6 or 10 carbon atoms,
Z represents hydrogen, represents optionally cyano-, halogen- or C₁-C₄-alkoxy-substituted alkyl having 1 to 6 carbon atoms, or represents in each case optionally cyano- or halogen-substituted alkenyl or alkinyl having, in each case, 3 to 6 carbon atoms.

3. Compounds of the formula (I) according to claim 1 or 2, characterized in that
n represents the numbers 0, 1 or 2,
A represents methylene, ethane-1,1-diyl (ethylidene), ethane-1,2-diyl (dimethylene), propane-1,1-diyl (propylidene), propane-1,2-diyl, propane-1,3-diyl (trimethylene), butane-1,2-diyl, butane-1,3-diyl or butane-1,4-diyl,
R² represents hydrogen, nitro, cyano, carboxyl, carbamoyl, thiocarbamoyl, fluorine, chlorine, bromine, iodine, or represents in each case optionally cyano-, fluorine-, chlorine-, methoxy-, ethoxy-, methylthio-, ethylthio-, methylsulphinyl-, ethylsulphinyl-, methylsulphonyl- or ethylsulphonyl-substituted methyl, ethyl, n- or i-propyl, n-, i-, s- or t-butyl, methoxy, ethoxy, n- or i-propoxy, n-, i-, s- or t-butoxy, methylthio, ethylthio, n- or i-propylthio, n-, i-, s- or t-butylthio, methylsulphinyl, ethylsulphinyl, n- or i-propylsulphinyl, methylsulphonyl, ethylsulphonyl, n- or i-propylsulphonyl, methylamino, ethylamino, n- or i-propylamino, n-, i-, s- or t-butylamino, dimethylamino, diethylamino, dimethylaminosulphonyl or diethylaminosulphonyl,
R³ represents hydrogen, nitro, cyano, carboxyl, carbamoyl, thiocarbamoyl, fluorine, chlorine, bromine, iodine, or represents in each case optionally cyano-, fluorine-, chlorine-, methoxy-, ethoxy-, methylthio-, ethylthio-, methylsulphinyl-, ethylsulphinyl- methylsulphonyl- or ethylsulphonyl-substituted methyl, ethyl, n- or i-propyl, n-, i-, s- or t-butyl, methoxy, ethoxy, n- or i-propoxy, n-, i-, s- or t-butoxy, methylthio, ethylthio, n- or i-propylthio, n-, i-, s- or t-butylthio, methylsulphinyl, ethylsulphinyl, n- or i-propylsulphinyl, methylsulphonyl, ethylsulphonyl, n- or i-propylsulphonyl, methylamino, ethylamino, n- or i-propylamino, n-, i-, s- or t-butylamino, dimethylamino, diethylamino, dimethylaminosulphonyl or diethylaminosulphonyl,
R⁴ represents methyl, ethyl, n- or i-propyl, chloromethyl, dichloromethyl, fluoromethyl, difluoromethyl, trifluoromethyl, chlorodifluoromethyl or fluorodichloromethyl,
m represents the numbers 0, 1, 2 or 3,
R⁵ represents fluorine, chlorine, bromine, represents in each case optionally fluorine- and/or chlorine-substituted methyl, ethyl, n- or i-propyl, n-, i-, s- or t-butyl, methylthio, ethylthio, n- or i-propylthio, n-, i-, s- or t-butylthio, or represents phenyl or—in the case where m represents 2—where appropriate also together with a second R⁵ radical represents oxygen, propane-1,3-diyl or butane-1,4-diyl,
R⁶ represents hydroxyl, formyloxy, fluorine, chlorine, represents in each case optionally fluorine- and/or chlorine-substituted methoxy, ethoxy, n- or i-propoxy, n-, i-, s- or t-butoxy, methylthio, ethylthio, n- or i-propylthio, n-, i-, s- or t-butylthio, methylsulphinyl, ethylsulphinyl, n- or i-propylsulphinyl, methylsulphonyl, ethylsulphonyl, n- or i-propylsulphonyl, acetyloxy, propionyloxy, n- or i-butyryloxy, methoxycarbonyloxy, ethoxycarbonyloxy, n- or i-propoxycarbonyloxy, methylaminocarbonyloxy, ethylaminocarbonyloxy, n- or i-propylaminocarbonyloxy, methylsulphonyloxy, ethylsulphonyloxy, n- or i-propylsulphonyloxy, represents in each case optionally fluorine-, chlorine- or bromine-substituted propenyloxy, butenyloxy, propinyloxy or butinyloxy, represents in each case optionally nitro-, cyano-, fluorine-, chlorine-, bromine-, methyl-, ethyl-, n- or i-propyl-, n-, i-, s- or t-butyl-, trifluoromethyl-, methoxy-, ethoxy-, n- or i-propoxy-, n-, i-, s- or t-butoxy-, difluoromethoxy-, trifluoromethoxy-, methylthio-, ethylthio-, n- or i-propylthio-, n-, i-, s- or t-butylthio-, difluoromethylthio-, trifluoromethylthio-, methylsulphinyl-, ethylsulphinyl-, n- or i-propylsulphinyl-, trifluoromethylsulphinyl-, methylsulphonyl-, ethylsulphonyl-, n- or i-propylsulphonyl- or trifluoromethylsulphonyl-substituted phenoxy, phenylthio, phenylsulphinyl, phenylsulphonyl, phenylcarbonyloxy, phenylcarbonylmethoxy, phenylsulphonyloxy, phenylmethoxy, phenylmethylthio, phenylmethylsulphinyl or phenylmethylsulphonyl,
R⁷ represents hydrogen, cyano, carbamoyl, thiocarbamoyl, fluorine, chlorine, bromine, represents in each case optionally fluorine- and/or chlorine-substituted methyl, ethyl, n- or i-propyl, n-, i-, s- or t-butyl, methoxy, ethoxy, n- or i-propoxy, n-, i-, s- or t-butoxy, methylthio, ethylthio, n- or i-propylthio, n-, i-, s- or t-butylthio, methylsulphinyl, ethylsulphinyl, n- or i-propylsulphinyl, methylsulphonyl, ethylsulphonyl, n- or i-propylsulphonyl, methoxycarbonyl, ethoxycarbonyl, n- or i-propoxycarbonyl, or represents in each case optionally cyano-, fluorine-, chlorine-, bromine-, methyl-, ethyl-, n- or i-propyl-substituted cyclopropyl, cyclobutyl, cyclopentyl or cyclohexyl,
R⁸ represents hydrogen, represents in each case optionally cyano-, fluorine-, chlorine-, methoxy- or ethoxy-substituted methyl, ethyl, n- or i-propyl, n-, i-, s- or t-butyl, represents in each case optionally cyano-, fluorine-, chlorine- or bromine-substituted propenyl, butenyl, propinyl or butinyl, represents in each case optionally cyano-, fluorine-, chlorine-, methyl-, ethyl-, n- or i-propyl-substituted cyclopropyl, cyclobutyl, cyclopentyl, cyclohexyl, cyclopropylmethyl, cyclobutylmethyl, cyclopentylmethyl or cyclohexylmethyl or represents in each case optionally nitro-, cyano-, fluorine-, chlorine-, bromine-, methyl-, ethyl-, n- or i-propyl-, n-, i-, s- or t-butyl-, trifluoromethyl-, methoxy-, ethoxy-, n- or i-propoxy-, n-, i-, s- or t-butoxy-, difluoromethoxy-, trifluoromethoxy-, methylthio-, ethylthio-, n- or i-propylthio-, n-, i-, s- or t-butylthio-, difluoromethylthio-, trifluoromethylthio-, methylsulphinyl-, ethylsulphinyl-, n- or i-propylsulphinyl-, trifluoromethylsulphinyl-, methylsulphonyl-, ethylsulphonyl-, n- or i-propylsulphonyl- or trifluoromethylsulphonyl-substituted phenyl, benzyl or phenylethyl,
R⁹ represents hydroxyl, formyloxy, represents in each case optionally cyano-, fluorine-, chlorine-, methoxy-, ethoxy-, n- or i-propoxy-substituted methoxy, ethoxy, n- or i-propoxy, n-, i-, s- or t-butoxy, acetyloxy, propionyloxy, n- or i-butyryloxy, methoxycarbonyloxy, ethoxycarbonyloxy, n- or i-propoxycarbonyloxy, methylaminocarbonyloxy, ethylaminocarbonyloxy, n- or i-propylaminocarbonyloxy, methylsulphonyloxy, ethylsulphonyloxy, n- or i-propylsulphonyloxy, represents in each case optionally cyano-, fluorine-, chlorine- or bromine-substituted propenyloxy, butenyloxy, propinyloxy or butinyloxy, or represents in each case optionally nitro-, cyano-, fluorine-, chlorine-, bromine-, methyl-, ethyl-, n- or i-propyl-, n-, i-, s- or t-butyl-, trifluoromethyl-, methoxy-, ethoxy-, n- or i-propoxy-, n-, i-, s- or t-butoxy-, difluoromethoxy-, trifluoromethoxy-, methylthio-, ethylthio-, n- or i-propylthio-, n-, i-, s- or t-butylthio-, difluoromethylthio-, trifluoromethylthio-, methylsulphinyl-, ethylsulphinyl-, n- or i-propylsulphinyl-, trifluoromethylsulphinyl-, methylsulphonyl-, ethylsulphonyl-, n- or i-propylsulphonyl- or trifluoromethylsulphonyl-substituted phenylmethoxy, phenylcarbonyloxy, phenylcarbonylmethoxy or phenylsulphonyloxy,
R¹⁰ represents hydrogen, cyano, carbamoyl, thiocarbamoyl, fluorine, chlorine, bromine, or represents in each case optionally cyano-, fluorine-, chlorine-, methoxy- or ethoxy-substituted methyl, ethyl, n- or i-propyl, n-, i-, s- or t-butyl, acetyl, propionyl, n- or i-butyryl, methoxy, ethoxy, n- or i-propoxy, n-, i-, s- or t-butoxy, methoxycarbonyl, ethoxycarbonyl, n- or i-propoxycarbonyl, methylthio, ethylthio, n- or i-propylthio, n- i-, s- or t-butylthio, methylsulphinyl, ethylsulphinyl, n- or i-propylsulphinyl, methylsulphonyl, ethylsulphonyl, n- or i-propylsulphonyl,
R¹¹ represents hydrogen, represents in each case optionally cyano-, fluorine-, chlorine-, methoxy- or ethoxy-substituted methyl, ethyl, n- or i-propyl, n-, i-, s- or t-butyl, or represents in each case optionally cyano-, fluorine-, chlorine-, methyl- or ethyl-substituted cyclopropyl, cyclobutyl, cyclopentyl or cyclohexyl,
R¹² represents hydrogen, represents in each case optionally cyano-, fluorine-, chlorine-, methoxy- or ethoxy-substituted methyl, ethyl, n- or i-propyl, n-, i-, s- or t-butyl, or represents in each case optionally cyano-, fluorine-, chlorine-, methyl- or ethyl-substituted cyclopropyl, cyclobutyl, cyclopentyl or cyclohexyl,
R¹³ represents hydrogen, cyano, carbamoyl, fluorine, chlorine, bromine, or represents in each case optionally cyano-, fluorine-, chlorine-, methoxy- or ethoxy-substituted methyl, ethyl, n- or i-propyl, n-, i-, s- or t-butyl, methoxy, ethoxy, n- or i-propoxy, n-, i-, s- or t-butoxy, methoxycarbonyl, ethoxycarbonyl, n- or i-propoxycarbonyl, methylthio, ethylthio, n- or i-propylthio, n-, i-, s- or t-butylthio, methylsulphinyl, ethylsulphinyl, n- or i-propylsulphinyl, methylsulphonyl, ethylsulphonyl, n- or i-propylsulphonyl,
Y¹ represents a single bond, O (oxygen), S (sulphur), N—Z, methylene or dimethylene (ethane-1,2-diyl),
Y⁴ represents cyano, nitro, represents in each case optionally fluorine- and/or chlorine-substituted acetyl, propionyl, n- or i-butyryl, methylsulphonyl, ethylsulphonyl, n- or i-propylsulphonyl, or represents in each case optionally nitro-, cyano-, fluorine-, chlorine-, bromine-, methyl-, ethyl-, n- or i-propyl-, n-, i-, s- or t-butyl-, trifluoromethyl-, methoxy-, ethoxy-, n- or i-propoxy-, difluoromethoxy- or trifluoromethoxy-substituted benzoyl or phenylsulphonyl,
Y⁵ represents hydrogen, cyano, nitro, represents in each case optionally fluorine- and/or chlorine-substituted acetyl, propionyl, n- or i-butyryl, methylsulphonyl, ethylsulphonyl, n- or i-propylsulphonyl, or represents in each case optionally nitro-, cyano-, fluorine-, chlorine-, bromine-, methyl-, ethyl-, n- or i-propyl-, n-, i-, s- or t-butyl-, trifluoromethyl-, methoxy-, ethoxy-, n- or i-propoxy-, difluoromethoxy- or trifluoromethoxy-substituted benzoyl or phenylsulphonyl,
Z represents hydrogen, represents in each case optionally cyano-, fluorine-, chlorine-, methoxy- or ethoxy-substituted methyl, ethyl, n- or i-propyl, n-, i-, s- or t-butyl, or represents in each case optionally cyano-, fluorine-, chlorine- or bromine-substituted propenyl, butenyl, propinyl or butinyl.

4. Compounds of the formula (1) according to any of claims 1 to 3, characterized in that
n represents the number 0,
A represents methylene or ethane-1,2-diyl (dimethylene),
R² represents hydrogen, nitro, cyano, fluorine, chlorine, bromine, iodine, methyl, ethyl, n- or i-propyl, difluoromethyl, trifluoromethyl, dichloromethyl, trichloromethyl, methoxymethyl, methylthiomethyl, methylsulphinylmethyl, methylsulphonylmethyl, methoxy, ethoxy, difluoromethoxy, trifluoromethoxy, methylthio, ethylthio, methylsulphinyl, ethylsulphinyl, methylsulphonyl, ethylsulphonyl or dimethylaminosulphonyl,
R³ represents hydrogen, nitro, cyano, fluorine, chlorine, bromine, iodine, methyl, ethyl, difluoromethyl, trifluoromethyl, dichloromethyl, trichloromethyl, methoxymethyl, methylthiomethyl, methylsulphinylmethyl, methylsulphonylmethyl, methoxy, ethoxy, difluoromethoxy, trifluoromethoxy, methylthio, ethylthio, methylsulphinyl, ethylsulphinyl, methylsulphonyl, ethylsulphonyl or dimethylaminosulphonyl,
m represents the numbers 0, 1 or 2,
R⁵ represents in each case optionally fluorine- and/or chlorine-substituted methyl, ethyl, n- or i-propyl, methylthio, ethylthio, n- or i-propylthio, or represents phenyl or—in the case where m represents 2—where appropriate also together with a second R⁵ radical represents propane-1,3-diyl or butane-1,4-diyl,
R⁶ represents hydroxyl, represents formyloxy, represents in each case optionally fluorine- and/or chlorine-substituted methoxy, ethoxy, n- or i-propoxy, methylthio, ethylthio, n- or i-propylthio, methylsulphinyl, ethylsulphinyl, methylsulphonyl, ethylsulphonyl, acetyloxy, propionyloxy, n- or i-butyryloxy, methoxycarbonyloxy, ethoxycarbonyloxy, n- or i-propoxycarbonyloxy, methylaminocarbonyloxy, ethylaminocarbonyloxy, n- or i-propylaminocarbonyloxy, methylsulphonyloxy, ethylsulphonyloxy, n- or i-propylsulphonyloxy, represents in each case optionally fluorine- or chlorine-substituted propenyloxy, butenyloxy, propinyloxy or butinyloxy, represents in each case optionally nitro-, cyano-, fluorine-, chlorine-, bromine-, methyl-, ethyl-, n- or i-propyl-, trifluoromethyl-, methoxy-, ethoxy-, n- or i-propoxy-, difluoromethoxy- or trifluoromethoxy-substituted phenoxy, phenylthio, phenylsulphinyl, phenylsulphonyl, phenylcarbonyloxy, phenylcarbonylmethoxy, phenylsulphonyloxy, phenylmethoxy, phenylmethylthio, phenylmethylsulphinyl or phenylmethylsulphonyl,
R⁷ represents hydrogen, cyano, fluorine, chlorine, bromine, represents in each case optionally fluorine- and/or chlorine-substituted methyl, ethyl, n- or i-propyl, methoxy, ethoxy, n- or i-propoxy, methylthio, ethylthio, n- or i-propylthio, methylsulphinyl, ethylsulphinyl, n- or i-propylsulphinyl, methylsulphonyl, ethylsulphonyl, n- or i-propylsulphonyl, methoxycarbonyl, ethoxycarbonyl, n- or i-propoxycarbonyl, or represents optionally cyano-, fluorine-, chlorine- or methyl-substituted cyclopropyl,
R⁸ represents hydrogen, represents in each case optionally cyano-, fluorine-, chlorine-, methoxy- or ethoxy-substituted methyl, ethyl, n- or i-propyl, n-, i-, s- or t-butyl, represents in each case optionally fluorine- or chlorine-substituted propenyl, butenyl, propinyl or butinyl, represents in each case optionally fluorine-, chlorine-, methyl- or ethyl-substituted cyclopropyl, or represents in each case optionally fluorine-, chlorine-, bromine-, methyl-, ethyl-, n- or i-propyl-, trifluoromethyl-, methoxy-, ethoxy-, n- or i-propoxy-, difluoromethoxy- or trifluoromethoxy-substituted phenyl or benzyl,
R⁹ represents hydroxyl, represents formyloxy, represents in each case optionally cyano-, fluorine-, chlorine-, methoxy-, ethoxy-, n- or i-propoxy-substituted methoxy, ethoxy, n- or i-propoxy, acetyloxy, propionyloxy, n- or i-butyryloxy, methoxycarbonyloxy, ethoxycarbonyloxy, n- or i-propoxycarbonyloxy, methylaminocarbonyloxy, ethylaminocarbonyloxy, n- or i-propylaminocarbonyloxy, methylsulphonyloxy, ethylsulphonyloxy, n- or i-propylsulphonyloxy, represents in each case optionally fluorine- or chlorine-substituted propenyloxy, butenyloxy, propinyloxy or butinyloxy, or represents in each case optionally nitro-, cyano-, fluorine-, chlorine-, bromine-, methyl-, ethyl-, n- or i-propyl-, trifluoromethyl- methoxy-, ethoxy-, n- or i-propoxy-, difluoromethoxy- or trifluoromethoxy-substituted phenylmethoxy, phenylcarbonyloxy, phenylcarbonylmethoxy or phenylsulphonyloxy,
R¹⁰ represents hydrogen, cyano, fluorine, chlorine, bromine, or represents in each case optionally fluorine-, chlorine-, methoxy- or ethoxy-substituted methyl, ethyl, n- or i-propyl, acetyl, propionyl, n- or i-butyryl, methoxy, ethoxy, n- or i-propoxy, methoxycarbonyl, ethoxycarbonyl, n- or i-propoxycarbonyl, methylthio, ethylthio, n- or i-propylthio, methylsulphinyl, ethylsulphinyl, methylsulphonyl or ethylsulphonyl,
R¹¹ represents hydrogen, represents in each case optionally fluorine-, chlorine-, methoxy- or ethoxy-substituted methyl, ethyl, n- or i-propyl, or represents optionally fluorine-, chlorine-, methyl- or ethyl-substituted cyclopropyl,
R¹² represents hydrogen, represents in each case optionally fluorine-, chlorine-, methoxy- or ethoxy-substituted methyl, ethyl, n- or i-propyl, n-, i-, s- or t-butyl, or represents optionally cyano-, fluorine-, chlorine-, methyl- or ethyl-substituted cyclopropyl,
R¹³ represents hydrogen, cyano, fluorine, chlorine, bromine, or represents in each case optionally fluorine-, chlorine-, methoxy- or ethoxy-substituted methyl, ethyl, n- or i-propyl, methoxy, ethoxy, n- or i-propoxy, methoxycarbonyl, ethoxycarbonyl, n- or i-propoxycarbonyl, methylthio, ethylthio, n- or i-propylthio, methylsulphinyl, ethylsulphinyl, n- or i-propylsulphinyl, methylsulphonyl, ethylsulphonyl, n- or i-propylsulphonyl,
Y¹ represents a single bond, O (oxygen), CH₂ (methylene) or N—Z,
Y³ represents N—Y⁴ or—in the case where Y¹ represents O (oxygen), S (sulphur) or N—Z—also represents O (oxygen) or S (sulphur), and
Z represents hydrogen, methyl, ethyl, n- or i-propyl.

5. Compounds of the formula (I) according to any of claims 1 to 4, characterized in that
R¹ represents one of the following groups

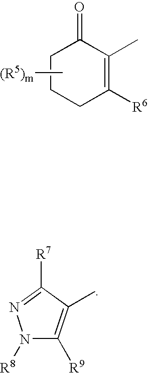

6. Compounds of the formula (I) according to any of claims 1 to 5, characterized in that
Y³ represents N—CN or—in the case where Y¹ represents O, S or N—Z—also represents O (oxygen).

7. Compounds of the formula (I) according to any of claims 1 to 6, characterized in that
Y³ represents N—CN.

8. Process for preparing compounds of the formula (I), characterized in that
(a) substituted benzoic acids of the formula (II) 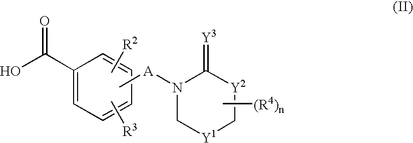
in which
n, A, R², R³, R⁴, Y¹, Y² and Y³ have the meaning indicated in claim 1,
or reactive derivatives thereof
are reacted with compounds of the general formula (III)
R¹—H (III)
in which
R¹ has the meaning indicated in claim, 1,
where appropriate in the presence of a dehydrating agent, where appropriate in the presence of one or more reaction auxiliaries, and where appropriate in the presence of one or more diluents,
or in that
(b) substituted benzoyl ketones of the general formula (Ia) 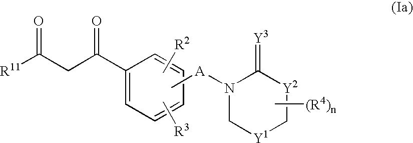
in which
n, A, R², R³, R⁴, R¹¹, Y¹, Y² and Y³ have the meanings indicated in claim 1,
are reacted with an orthoformic ester or with an N,N-dimethyl-formamide acetal or with a cyanoformic ester or with carbon disulphide and an alkylating agent and subsequently with hydroxylamine or an acid adduct thereof
where appropriate in the presence of one or more reaction auxiliaries and where appropriate in the presence of one or more diluents,
and, where appropriate, after processes (a) or (b) have been carried out, are subjected the compounds of the general formula (I) which have been obtained in this way to substitution, oxidation or reduction reactions and/or the compounds of the general formula (I) are converted into salt-like compounds in a conventional way.

9. Compounds of the formula (II) 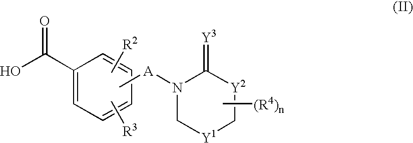
in which
n, A, R², R³, R⁴, Y¹, Y² and Y³ have the meaning indicated in claim 1.

10. Process for preparing compounds of the formula (II) according to claim 9, characterized in that
(α) halogenoalkylbenzoic esters of the general formula (IV) 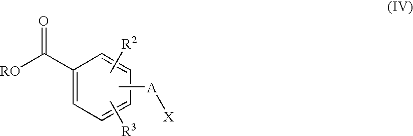
in which
A, R² and R³ have the meaning indicated in claim 1,
R represents alkyl, and
X represents halogen or alkylsulphonyloxy,
are reacted with compounds of the general formula (V) 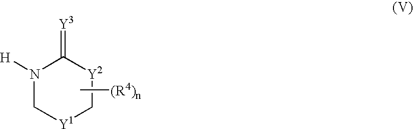
in which
n, R⁴, Y¹, Y² and Y³ have the meaning indicated in claim 1,
where appropriate in the presence of an acid acceptor and where appropriate in the presence of a diluent at temperatures between 0° C. and 100° C.,
or in that
in the case of compounds of the formula (II) in which Y¹ represents O (oxygen) or N—Z and Y² represents N—Z,
(β) halogenoalkylbenzoic esters of the formula (IV) 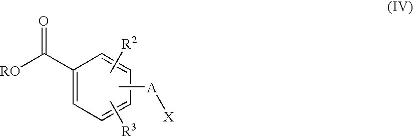
in which
A, R, R², R³ and X have the meaning indicated in claim 1 and claim 10
are reacted in a first step with compounds of the general formula (VI) 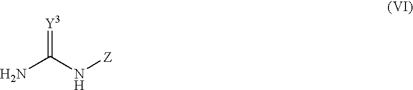
in which
Y³ and Z have the meaning indicated in claim 1,
where appropriate in the presence of an acid acceptor and where appropriate in the presence of a diluent at temperatures between 0° C. and 1 00° C., and the ureidoalkylbenzoic esters of the general formula (VII) which are formed thereby 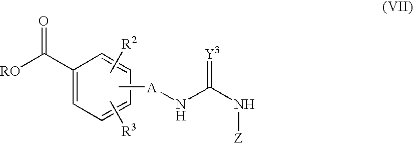
in which
A, R, R², R³, Y³ and Z have the meaning indicated in claim 1,
are reacted with formaldehyde or paraformaldehyde, where appropriate in the presence of a reaction auxiliary and where appropriate in the presence of one or more diluents and where appropriate with an amine of the general formula H₂N—Z at temperatures between 0° C. and 150° C.,
and, where appropriate, the substituted benzoic esters of the general formula (IIa) obtained by process variants (α) or (β) 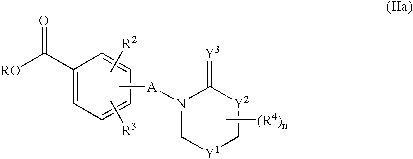
in which
n, A, R², R³, R⁴, Y¹, Y² and Y³ have the meaning indicated in claim 1,
and
R represents alkyl,
are hydrolysed in a conventional way by reacting with water in the presence of a base and, where appropriate, in the presence of one or more diluents at temperatures between 0° C. and 100° C.

11. Compounds of the formula (Ia) 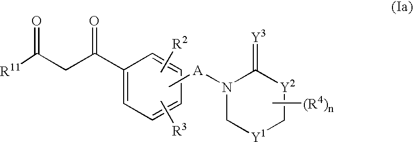
in which
n, A, R², R³, R⁴, R¹¹, Y¹, Y² and Y³ have the meaning indicated in claim 1.

12. Herbicidal compositions characterized by a content of at least one compound of the formula (I) according to claim 1.

13. Use of one or more compounds of the formula (I) according to claim 1 for controlling weeds.

14. Method for controlling weeds, characterized in that at least one compound of the formula (I) according to claim 1 is allowed to act on weeds and/or their habitat.

15. Use of compositions according to claim 12 for controlling weeds.
